SEQUENCE LISTING (53 sequences)

SEQ ID NO: 1 - DNA, 60 bp, synthetic construct
  source 1..60: mol_type = other DNA
gtctcgtgggctcggtccgactagatgtgtataagagacagttccctaccatggtgttcc

SEQ ID NO: 2 - DNA, 60 bp, synthetic construct
  source 1..60: mol_type = other DNA
tcgtcggcagcgtctccgactagatgtgtataagagacaggtgtaggtttaggggctggt

SEQ ID NO: 3 - DNA, 41 bp, synthetic construct
  source 1..41: mol_type = other DNA
gtctcgtgggctcggtccgactagatgtgtataagagacag

SEQ ID NO: 4 - DNA, 40 bp, synthetic construct
  source 1..40: mol_type = other DNA
tcgtcggcagcgtctccgactagatgtgtataagagacag

SEQ ID NO: 5 - DNA, 19 bp, synthetic construct
  source 1..19: mol_type = other DNA
  misc_feature 1: note = /5Phos/
ctgtctcttatacacatct

SEQ ID NO: 6 - DNA, 34 bp, synthetic construct
  source 1..34: mol_type = other DNA
gtctcgtgggctcggagatgtgtataagagacag

SEQ ID NO: 7 - DNA, 33 bp, synthetic construct
  source 1..33: mol_type = other DNA
tcgtcggcagcgtcagatgtgtataagagacag

SEQ ID NO: 8 - DNA, 27 bp, synthetic construct
  source 1..27: mol_type = other DNA
  misc_feature 1: note = /5Biosg/
gacttactgaagatctacagcagtgag

SEQ ID NO: 9 - DNA, 25 bp, synthetic construct
  source 1..25: mol_type = other DNA
  misc_feature 1: note = /5Phos/
cactgctgtagatcttcagtaagtc

SEQ ID NO: 10 - DNA, 27 bp, synthetic construct
  source 1..27: mol_type = other DNA
  misc_feature 1: note = /5Biosg/
cgatctctgaagcattgcagcagcgag

SEQ ID NO: 11 - DNA, 25 bp, synthetic construct
  source 1..25: mol_type = other DNA
  misc_feature 1: note = /5Phos/
cgctgctgcaatgcttcagagatcg

SEQ ID NO: 12 - DNA, 58 bp, synthetic construct
  source 1..58: mol_type = other DNA
gtgactggagttcagacgtgtgctcttccgatctcgatctctgaagcattgcagcagc

SEQ ID NO: 13 - DNA, 60 bp, synthetic construct
  source 1..60: mol_type = other DNA
acactctttccctacacgacgctcttccgatctgacttactgaagatctacagcagtgag

SEQ ID NO: 14 - DNA, 57 bp, synthetic construct
  source 1..57: mol_type = other DNA
acactctttccctacacgacgctcttccgatctcgatctctgaagcattgcagcagc

SEQ ID NO: 15 - DNA, 20 bp, synthetic construct
  source 1..20: mol_type = other DNA
  misc_feature 1: note = /5Phos/
ggctcacatccgccgttgtc

SEQ ID NO: 16 - DNA, 18 bp, synthetic construct
  source 1..18: mol_type = other DNA
gacaacggcggatgtgag

SEQ ID NO: 17 - DNA, 52 bp, synthetic construct
  source 1..52: mol_type = other DNA
gtgactggagttcagacgtgtgctcttccgatctgacaacggcggatgtgag

SEQ ID NO: 18 - DNA, 57 bp, synthetic construct
  source 1..57: mol_type = other DNA
  misc_feature 1: note = /5Phos/
gtttaagagctatgctggaaacagcatagcaagtttaaataaggctagtccgttatc

SEQ ID NO: 19 - DNA, 59 bp, synthetic construct
  source 1..59: mol_type = other DNA
gataacggactagccttatttaaacttgctatgctgtttccagcatagctcttaaacnn

SEQ ID NO: 20 - DNA, 59 bp, synthetic construct
  source 1..59: mol_type = other DNA
gtgactggagttcagacgtgtgctcttccgatctgataacggactagccttatttaaac

SEQ ID NO: 21 - DNA, 58 bp, synthetic construct
  source 1..58: mol_type = other DNA
cttgaaagtatttcgatttcttggctttatatatcttgtggaaaggacgaaacaccgn

SEQ ID NO: 22 - DNA, 56 bp, synthetic construct
  source 1..56: mol_type = other DNA
  misc_feature 1: note = /5Phos/
ggtgtttcgtcctttccacaagatatataaagccaagaaatcgaaatactttcaag

SEQ ID NO: 23 - DNA, 57 bp, synthetic construct
  source 1..57: mol_type = other DNA
acactctttccctacacgacgctcttccgatctcttgaaagtatttcgatttcttgg

SEQ ID NO: 24 - DNA, 25 bp, synthetic construct
  source 1..25: mol_type = other DNA
  misc_feature 1: note = /5Biosg/
gacttactgaagatctacagcagtg

SEQ ID NO: 25 - DNA, 20 bp, synthetic construct
  source 1..20: mol_type = other DNA
  misc_feature 1: note = /5Biosg/
cgatctctgaagcattgcag

SEQ ID NO: 26 - DNA, 47 bp, synthetic construct
  source 1..47: mol_type = other DNA
caagcagaagacggcatacgagatcgagtaatgtctcgtgggctcgg

SEQ ID NO: 27 - DNA, 47 bp, synthetic construct
  source 1..47: mol_type = other DNA
caagcagaagacggcatacgagattctccggagtctcgtgggctcgg

SEQ ID NO: 28 - DNA, 47 bp, synthetic construct
  source 1..47: mol_type = other DNA
caagcagaagacggcatacgagataatgagcggtctcgtgggctcgg

SEQ ID NO: 29 - DNA, 51 bp, synthetic construct
  source 1..51: mol_type = other DNA
aatgatacggcgaccaccgagatctacactatagccttcgtcggcagcgtc

SEQ ID NO: 30 - DNA, 51 bp, synthetic construct
  source 1..51: mol_type = other DNA
aatgatacggcgaccaccgagatctacacatagaggctcgtcggcagcgtc

SEQ ID NO: 31 - DNA, 51 bp, synthetic construct
  source 1..51: mol_type = other DNA
aatgatacggcgaccaccgagatctacaccctatccttcgtcggcagcgtc

SEQ ID NO: 32 - DNA, 47 bp, synthetic construct
  source 1..47: mol_type = other DNA
caagcagaagacggcatacgagatggaatctcgtctcgtgggctcgg

SEQ ID NO: 33 - DNA, 47 bp, synthetic construct
  source 1..47: mol_type = other DNA
caagcagaagacggcatacgagatttctgaatgtctcgtgggctcgg

SEQ ID NO: 34 - DNA, 47 bp, synthetic construct
  source 1..47: mol_type = other DNA
caagcagaagacggcatacgagatacgaattcgtctcgtgggctcgg

SEQ ID NO: 35 - DNA, 47 bp, synthetic construct
  source 1..47: mol_type = other DNA
caagcagaagacggcatacgagatagcttcaggtctcgtgggctcgg

SEQ ID NO: 36 - DNA, 70 bp, synthetic construct
  source 1..70: mol_type = other DNA
aatgatacggcgaccaccgagatctacactatagcctacactctttccctacacgacgctcttccgatct

SEQ ID NO: 37 - DNA, 70 bp, synthetic construct
  source 1..70: mol_type = other DNA
aatgatacggcgaccaccgagatctacacatagaggcacactctttccctacacgacgctcttccgatct

SEQ ID NO: 38 - DNA, 70 bp, synthetic construct
  source 1..70: mol_type = other DNA
aatgatacggcgaccaccgagatctacaccctatcctacactctttccctacacgacgctcttccgatct

SEQ ID NO: 39 - DNA, 70 bp, synthetic construct
  source 1..70: mol_type = other DNA
aatgatacggcgaccaccgagatctacacggctctgaacactctttccctacacgacgctcttccgatct

SEQ ID NO: 40 - DNA, 70 bp, synthetic construct
  source 1..70: mol_type = other DNA
aatgatacggcgaccaccgagatctacacaggcgaagacactctttccctacacgacgctcttccgatct

SEQ ID NO: 41 - DNA, 70 bp, synthetic construct
  source 1..70: mol_type = other DNA
aatgatacggcgaccaccgagatctacactaatcttaacactctttccctacacgacgctcttccgatct

SEQ ID NO: 42 - DNA, 66 bp, synthetic construct
  source 1..66: mol_type = other DNA
caagcagaagacggcatacgagatcgagtaatgtgactggagttcagacgtgtgctcttccgatct

SEQ ID NO: 43 - DNA, 66 bp, synthetic construct
  source 1..66: mol_type = other DNA
caagcagaagacggcatacgagattctccggagtgactggagttcagacgtgtgctcttccgatct

SEQ ID NO: 44 - DNA, 66 bp, synthetic construct
  source 1..66: mol_type = other DNA
caagcagaagacggcatacgagataatgagcggtgactggagttcagacgtgtgctcttccgatct

SEQ ID NO: 45 - DNA, 66 bp, synthetic construct
  source 1..66: mol_type = other DNA
caagcagaagacggcatacgagatggaatctcgtgactggagttcagacgtgtgctcttccgatct

SEQ ID NO: 46 - DNA, 66 bp, synthetic construct
  source 1..66: mol_type = other DNA
caagcagaagacggcatacgagatttctgaatgtgactggagttcagacgtgtgctcttccgatct

SEQ ID NO: 47 - DNA, 66 bp, synthetic construct
  source 1..66: mol_type = other DNA
caagcagaagacggcatacgagatacgaattcgtgactggagttcagacgtgtgctcttccgatct

SEQ ID NO: 48 - DNA, 66 bp, synthetic construct
  source 1..66: mol_type = other DNA
caagcagaagacggcatacgagatagcttcaggtgactggagttcagacgtgtgctcttccgatct

SEQ ID NO: 49 - DNA, 66 bp, synthetic construct
  source 1..66: mol_type = other DNA
caagcagaagacggcatacgagatgcgcattagtgactggagttcagacgtgtgctcttccgatct

SEQ ID NO: 50 - DNA, 200 bp, synthetic construct
  source 1..200: mol_type = other DNA
tccctaccatggtgttccccttcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggccccaccagcccctaaacctaca

SEQ ID NO: 51 - DNA, 17 bp, synthetic construct
  source 1..17: mol_type = other DNA
gtctcgtgggctcggtc

SEQ ID NO: 52 - DNA, 16 bp, synthetic construct
  source 1..16: mol_type = other DNA
tcgtcggcagcgtctc

SEQ ID NO: 53 - DNA, 200 bp, synthetic construct
  source 1..200: mol_type = other DNA
tccctaccatggtgttccccggcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggccccaccagcccctaaacctaca

(19) United States
(12) Patent Application Publication
(10) Pub. No.: US 2024/0409922 A1
(43) Pub. Date: Dec. 12, 2024

(54) METHOD OF GENERATING A LIBRARY OF POLYNUCLEOTIDE MOLECULES ENCODING GUIDE RNAS

(71) Applicant: GENOME RESEARCH LIMITED, Cambridgeshire (GB)

(72) Inventors: Qianxin WU, Cambridgeshire (GB); Ashutosh TREHAN, Cambridgeshire (GB)

(21) Appl. No.: 18/698,350

(22) PCT Filed: Oct. 4, 2022
(86) PCT No.: PCT/GB2022/052509

§ 371 Date: Apr. 3, 2024

(30) Foreign Application Priority Data
Oct. 4, 2021 (GB) 2114206.2

(51) Int. Cl. C12N 15/10

(52) U.S. Cl. CPC C12N 15/1093

(57) ABSTRACT

The invention relates to a method of generating a library of polynucleotide molecules encoding guide RNAs (gRNAs) from target polynucleotide(s). The invention also relates to a library of polynucleotide molecules encoding gRNAs obtainable by the aforementioned method, and a gRNA library generation kit thereof.

### FIELD OF THE INVENTION

The invention relates to a method of generating a library of polynucleotide molecules encoding guide RNAs (gRNAs) from target polynucleotide(s). The invention also relates to a library of polynucleotide molecules encoding gRNAs obtainable by the aforementioned method, and a gRNA library generation kit thereof.

### BACKGROUND OF THE INVENTION

Genome-wide clustered regularly interspaced short palindrome repeats (CRISPR)-Cas knockout screens are used to study the relationship between genotype and phenotype by knocking-out gene expression on a genome-wide scale and studying the resulting phenotypic changes. The approach uses the CRISPR-Cas gene editing system, coupled with libraries of oligonucleotides called guide RNAs (gRNAs). The Cas nuclease is directed by the gRNA that is complementary with the target DNA strand. In the presence of a short sequence called a protospacer-adjacent motif (PAM) on the opposite DNA strand, the gRNA binds to the target strand by complementarity and guides Cas to generate site-specific double-strand breaks (DSBs) on the target DNA sequence. The resultant DSBs are then processed by DNA repair mechanisms such as non-homologous end joining (NHEJ) or homology directed repair (HDR). HDR can result in precise gene replacement whereas NHEJ is error prone in that it may induce frameshift indel mutations that can abolish target gene function.

Genome-wide CRISPR screens have revolutionized prioritization of novel drug targets in both academia and industry. However, oligonucleotide design and synthesis for whole genome CRISPR screens is resource intensive and costly. Consequently, most human genome-wide CRISPR screen studies use universal libraries generated by a handful of labs around the world and are restricted to protein coding regions of the reference genome. The rapidly growing field of genome-wide association studies (GWAS) suggests that the majority of single nucleotide polymorphisms (SNPs) linked to disease fall within the non-coding genome; however, tools to systematically screen the non-coding genome for functionality are limited.

There is therefore a need for methods which can generate patient and cell type specific gRNA libraries targeting functional coding and non-coding genome in a high-throughput and cost-effective manner.

### SUMMARY OF THE INVENTION

According to a first aspect of the invention, there is provided a method of generating a library of polynucleotide molecules encoding guide RNAs (gRNAs) from target polynucleotide(s) comprising incubation of the target polynucleotide(s) with insertional enzyme complexes, wherein each of said insertional enzyme complexes comprises (i) an insertional enzyme and (ii) one or more tagmentation adapters to generate a plurality of tagged cleavage fragments.

According to a further aspect of the invention there is provided a library of polynucleotide molecules encoding guide RNAs (gRNAs) obtainable by the methods as disclosed herein.

According to a further aspect of the invention there is provided a guide RNA (gRNA) library generation kit which comprises:
(a) an insertional enzyme;
(b) a plurality of tagmentation adapters which comprise a first restriction site;
(c) a restriction enzyme which recognises the first restriction site;
(d) a plurality of first ligation adapters which comprise a second restriction site, a third restriction site, and a label;
(e) a restriction enzyme which recognises the second restriction site;
(f) a plurality of second ligation adapters which comprise a fourth restriction site and part or all of a protospacer adjacent motif (PAM) sequence;
(g) a restriction enzyme which recognises the fourth restriction site;
(h) a restriction enzyme which recognises the third restriction site; and
(i) a plurality of third and fourth ligation adapters which comprise vector cloning sequences.

### BRIEF DESCRIPTION OF THE FIGURES

FIG. 1: A representative method of generating the claimed library of polynucleotides encoding Cas guide RNAs (gRNAs). A target polynucleotide is first incubated with an insertional enzyme (eg. transposome) to generate polynucleotide fragments that are flanked by adapters containing sequences for first restriction enzyme (RE) site, mosaic end (ME) and primer binding sites. After PCR amplification, the sequences are digested with the first restriction enzyme (RE1) and then ligated with adapter1 using a ligase. The adapter 1 contains a label (e.g. biotin) and sequences recognised by restriction enzyme 2 (RE2) and restriction enzyme 3 (RE3). After adapter1 ligation, the sequences are digested with RE2 and subsequently ligated with adapter 2. The adapter 2 contains restriction enzyme 4 (RE4) binding site and a flanking sequence containing either part of or the whole PAM sequence. This is followed by RE4 digestion to remove the PAM and adapter 2 sequences. The RE4 digested product is then ligated with adapter 3. The resulting sequences are then digested with RE3 to remove adapter 1 sequences, followed by adapter 4 ligation. Both adapter 3 and adapter 4 may contain additional RE sites, sequences for PCR amplification and cloning into a vector of choice.

FIG. 2: Tn5 Purification and tagmentation of genomic DNA (gDNA). (A) Tn5 was loaded with TAG-Adapt1-i7-FW/MEDS-REV and TAG-Adapt1-i5-FW/MEDS-REV adapters (referred hereafter as custom adapters) on chitin magnetic beads and after purification a 55 kD band was observed. (B) Tn5 was also assembled in solution with either custom adapters or standard nextera adapters (MEDS-A/MEDS-REV and MEDS-B/MEDS-REV) and tagmentation of gDNA was observed in both the cases. This demonstrated that the modification (RE1 site insertion for Mmel restriction enzyme) introduced in custom adapters had no effect on the ability of Tn5 to tagment gDNA when compared to Tn5 loaded with standard Nextera adapters. (C) Similarly, Tn5 assembled on magnetic beads with custom adapters could also tagment gDNA.

FIG. 3: NGS library preparation of different steps for gDNA and defined template (of known sequence as a positive control). After each adapter ligation (Adapter 1-4) of gDNA and defined template, the sample was PCR amplified (5-12 cycles) to add multiplexing indexes and P5/P7 sites for Illumina sequencing. The control samples had the DNA template but no PCR amplification was performed. The expected sizes of the PCR products are mentioned under the 2% agarose gel image. The expected bands were observed after ligation of Adapter 1 (A), Adapter 2 (B), Adapter 3 (C) and Adapter 4 (D). Some extra bands, in addition to the right one, were also observed for Adapter 3 ligated products (C), probably due to PCR amplification bias.

FIG. 4: Adapter 2 ligation of gDNA sample. (A) Representative alignment of sequences are shown after adapter 2 ligation of gDNA sample. The left side shows the correct orientation with adapter 1 sequences containing second and third restriction enzyme site (EcoP15I and Acul, respectively) followed by 21 bp of varying sequences (NNNNNNNNNNNNNNNNNNNNN) and the right side contains the fourth restriction enzyme site (Ecil) from adapter 2 ligation. N can be any nucleotide A, T, G or C. (B-D) The sequences upon alignment to the genome shows 21 bp sequences (highlighted in dark gray) followed by either GG of the NGG PAM or CC sequence on the other strand (highlighted in light gray), as expected.

FIG. 5. Adapter 3 ligation of gDNA sample. (A) Representative sequences are aligned after ligation of adapter 3. The left side shows sequences from adapter 1, followed by 20 bp of varying sequences and the right side shows sequences from adapter 3 ligation. (B-C) Upon alignment of the 20 bp middle sequence to the genome (highlighted in dark gray), the adjoining sequence is either NGG or NCC on the reverse strand (highlighted in light gray), as expected.

FIG. 6: Adapter 4 ligation of gDNA sample. (A) Representative sequences aligned after Adapter 4 ligation. The left side shows correct sequences derived from adapter 4 ligation followed by 20 bp of varying sequences representing the gRNA. The right side shows correct sequences derived from Adapter 3. (B-E) Upon alignment of 20 gRNA sequence to the genome (highlighted in dark gray), the adjoining PAM sequences can be found as NGG or NCC on the reverse strand (highlighted in light gray).

FIG. 7: (A) Schematic of defined template showing NGG only on one end. The defined template was designed to ensure when it is cut by RE2, the end with NGG should proceed with the subsequent steps of the method. (B) We could clearly observe that the defined template upon undergoing all the steps has a clear NGG bias in the sequences that were observed after sequencing.

FIG. 8: NGS library preparation of different steps for DNA derived from chromatin of a cell line (K562), labelled as ATAC, genomic DNA (gDNA) and defined templates (of known sequence as a positive controls). After each adapter ligation (Adapter 1-4) of ATAC, gDNA and defined templates (template 1 and template 2), the sample was PCR amplified to add multiplexing indexes and P5/P7 sites for Illumina sequencing. The expected sizes of the PCR products are mentioned under the 2% agarose gel image. The expected bands were observed after ligation of Adapter 1 (A), Adapter 2 (B), Adapter 3 (C) and Adapter 4 (D). (E) ATAC sample after PCR amplification was run on Bioanalyzer to see the banding pattern at regular intervals.

FIG. 9: Characterization of Adapter 4 sequencing reads. (A) Most of the sequencing reads after adapter trimming are 17-25 bp long, as expected. (B-E) Length distribution of 17-25 bp filtered reads shows majority of the reads are 20 bp in length for ATAC (B), genomic DNA (C), template 2 (D) and template 1 (E).

FIG. 10: (A-B). Majority of the filtered sequencing reads (17-25 bp) for Adapter 4can be aligned back to the human genome (hg38) for gDNA and ATAC. (C) The percentage of reads that intersect with DNAse hypersensitivity regions demonstrates ATAC reads are 12-fold enriched as compared to gDNA. (D-F). ATAC reads (20 bp) when aligned to the genome demonstrates the presence of NGG sequences next to them (shaded region).

### DETAILED DESCRIPTION OF THE INVENTION

Disclosed herein is customized ATAC based CRISPR (cus-ATAC-CRISPR), which is a method of using insertional enzymes to generate patient and cell-type specific gRNA libraries in a high-throughput and cost-effective manner. Both promoters and enhancers of transcriptionally active genes display increased sensitivity to nuclease digestion and are located to “open” chromatin domains. Assay for Transposase Accessible Chromatin using sequencing (ATAC-seq) is a method of probing open chromatin based on the ability of transposases such as Tn5 transposase and MuA transposase to fragment DNA (described in US20160060691A1). Therefore, by using insertional enzymes such as Tn5 transposase to generate a gRNA library from genomic DNA, the gRNAs should target all functionally “active” regions of the genome, including non-coding regions, and will carry the unique genetic variants (such as single nucleotide polymorphisms (SNPs), short insertions/deletions, and structural variants) from the individual cells and patients. In addition, the reactions can be carried out using any transposase accessible DNA as a substrate at a comparatively lower cost. Importantly, cus-ATAC-CRISPR does not require prior knowledge of sequence or ATAC profiles, could easily be applied to less well sequenced species or novel contexts, and will target all regions of open chromatin, including the non-coding genome.

Thus, according to a first aspect of the invention, there is provided a method of generating a library of polynucleotide molecules encoding guide RNAs (gRNAs) from target polynucleotide(s) comprising incubation of the target polynucleotide(s) with insertional enzyme complexes, wherein each of said insertional enzyme complexes comprises (i) an insertional enzyme and (ii) one or more tagmentation adapters to generate a plurality of tagged cleavage fragments.

Reference herein to “guide RNAs” or “gRNAs” is intended to refer to any RNA molecule that recognises a target polynucleotide region of interest and directs a polynucleotide-targeting enzyme to that region. In some embodiments, the polynucleotide-targeting enzyme is a DNA-targeting enzyme. In some embodiments the DNA-targeting enzyme is a Cas endonuclease. In some embodiments the Cas endonuclease is Cas9 endonuclease. In another embodiment, the Cas endonuclease is Cas12a or a variant thereof, such as AsCas12a (from Acidaminococcus sp. BV3L6), LbCas12a (from Lachnospiraceae bacterium ND 2006), CeCas12a (from Coprococcus eutactus) or FnCas12a. In a still further embodiment, the DNA-targeting enzyme is a Cas variant, such as xCas9, SpCas9-NG, SpG or SpRY. In an alternative embodiment, the DNA-targeting enzyme is RNA guided DNA endonuclease. In some embodiments, the gRNA is a single guide RNA (sgRNA). The sgRNA is an RNA molecule consisting of two parts, a ‘constant region’ and a ‘variable region’. The constant region is the tracer RNA (tracrRNA), also referred to as the scaffold sequence, and is the sequence that binds to the Cas endonuclease. The variable region of the sgRNA is the CRISPR RNA (crRNA) which contains a sequence specific to the region of interest. In further embodiments, the sgRNA is a Cas9 sgRNA. For other Cas enzymes such as Cas12a the gRNA includes just the crRNA and not the tracrRNA.

The crRNA sequence is typically about 20 nucleotides in length. In some embodiments, the crRNA sequence is 17 nucleotides in length. In some embodiments, the crRNA sequence is 18 nucleotides in length. In some embodiments, the crRNA sequence is 19 nucleotides in length. In some embodiments, the crRNA sequence is 20 nucleotides in length. In some embodiments, the crRNA sequence is 21 nucleotides in length. In some embodiments the crRNA sequence is 22 nucleotides in length. In some embodiments, the crRNA sequence is 23 nucleotides in length. In some embodiments, the crRNA sequence is 24 nucleotides in length. In some embodiments, the crRNA sequence is 25 nucleotides in length. Thus, in some embodiments the sgRNA sequence is 17 nucleotides in length. In some embodiments, the sgRNA sequence is 18 nucleotides in length. In some embodiments, the sgRNA sequence is 19 nucleotides in length. In some embodiments, the sgRNA sequence is 20 nucleotides in length. In some embodiments, the sgRNA sequence is 21 nucleotides in length. In some embodiments the sgRNA sequence is 22 nucleotides in length. In some embodiments, the sgRNA sequence is 23 nucleotides in length. In some embodiments, the sgRNA sequence is 24 nucleotides in length. In some embodiments, the sgRNA sequence is 25 nucleotides in length.

In some embodiments, the crRNA sequence is at least 17 nucleotides in length. In some embodiments, the crRNA sequence is at least 18 nucleotides in length. In some embodiments, the crRNA sequence is at least 19 nucleotides in length. In some embodiments, the crRNA sequence is at least 20 nucleotides in length. In some embodiments, the crRNA sequence is at least 21 nucleotides in length. In some embodiments the crRNA sequence is at least 22 nucleotides in length. In some embodiments, the crRNA sequence is at least 23 nucleotides in length. In some embodiments, the crRNA sequence is at least 24 nucleotides in length. In some embodiments, the crRNA sequence is at least 25 nucleotides in length. Thus, in some embodiments the sgRNA sequence is at least 17 nucleotides in length. In some embodiments, the sgRNA sequence is at least 18 nucleotides in length. In some embodiments, the sgRNA sequence is at least 19 nucleotides in length. In some embodiments, the sgRNA sequence is at least 20 nucleotides in length. In some embodiments, the sgRNA sequence is at least 21 nucleotides in length. In some embodiments the sgRNA sequence is at least 22 nucleotides in length. In some embodiments, the sgRNA sequence is at least 23 nucleotides in length. In some embodiments, the sgRNA sequence is at least 24 nucleotides in length. In some embodiments, the sgRNA sequence is at least 25 nucleotides in length.

In some embodiments, Cas9 comprises an inactive or catalytically dead Cas9 variant (also known as dCas9). This variant is known to inhibit transcription by blocking either initiation or elongation by the RNA polymerase complex. Furthermore, dCas9 activator can be used to increase transcription by recruiting transcription factor or modify chromatin modification complex. Reference herein to “target polynucleotide(s)” is intended to refer to any polynucleotide or collection of polynucleotides (e.g., a chromosome, a collection of chromosomes, reverse transcribed RNA or RNA-DNA duplex, PCR-amplified DNA, targeted capturing sequences etc.) from which gRNAs are to be generated.

Examples of the types of suitable target polynucleotides include but are not limited to: chromosomal DNA (e.g., a chromosome, a genome, a collection of chromosomes), viral DNA, unknown DNA collected from any source (e.g., collected from an environmental source), DNA from an organelle (mitochondrial DNA, nuclear DNA, chloroplast DNA, and the like). Examples of suitable cellular sources for the target polynucleotide(s) include, but are not limited to: a eukaryotic cell; a prokaryotic cell, (e.g., a bacterial cell or an archaeal cell), a cell of a single-cell eukaryotic organism; a plant cell (e.g., rice, soy, maize, corn, wheat, tomato, tobacco, fruit tree, etc.); an algal cell (e.g., Botryococcus braunii, Chlamydomonas reinhardtii, Nannochloropsis gaditana, Chlorella pyrenoidosa, Sargassum patens C. Agardh, and the like); a fungal cell (e.g., a yeast cell); an animal cell; a cell from an invertebrate animal (e.g. fruit fly, cnidarian, echinoderm, nematode, planarian, etc.); a cell from a vertebrate animal (e.g., fish, e.g., zebrafish, amphibian, e.g, frog, reptile, bird, e.g., chicken, mammal, and the like); a cell from a mammal (e.g., zoo animal, pet, canine, equine, porcine, rodent, primate, human, etc.); and the like. The cellular source may be a single cellular source or a pooled sample of multiple cellular sources. The target polynucleotide may also be prepared from RNA. For example, complementary DNA (cDNA) may be synthesized from a single-stranded RNA template in a reaction catalysed by the reverse transcriptase enzyme. The reverse transcription of RNA into cDNA may also be combined with the amplification of specific DNA targets using reverse transcription polymerase chain reaction (RT-PCR) and insertional enzymatic reaction using dsDNA or DNA-RNA duplex. In order to generate a plurality of tagged cleavage fragments, the target polynucleotides must be accessible to the insertional enzyme complexes. For example, wherein the target polynucleotide is chromosomal DNA, target polynucleotides are accessible when chromatin is open such as in regions of the genome which are undergoing active transcription. Accessibility is similarly required wherein the target polynucleotide is viral DNA, organelle-derived DNA or DNA from another source which may be packaged.

Reference herein to a “PAM sequence” or “protospacer adjacent motif sequence” is intended to refer to a 2-8 base pair DNA sequence which is recognized by a genome editing enzyme such as an RNA-guided DNA endonuclease (e.g., a Cas9, Mad7, or Cpf1 endonuclease) to promote cleavage of the target site by the endonuclease. For example, the Cas9 endonuclease from Streptococcus pyogenes recognizes the PAM sequence 5′-NGG-3′ (where “N” can be any nucleotide base). In other examples, SpG has been shown to recognize the PAM sequence 5′-NGN-3′, and SpRY can target almost all PAMs in the genome (with 5′-NRN-3′ being preferable to 5′-NYN-3′, where R is A or G and Y is C or T; Liang et al. (2022) Nat Comm., 13 (3421), doi: https://doi.org/10.1038/s41467-022-31034-8). xCas9 displays one of the broadest PAM compatibility profiles, recognizing NG, NNG, GAT, CAA NG (A/G/T), (G/C) AG and (G/C/T) GCC PAMs (Hu et al. (2018) Nature, doi: 10.1038/nature26155), while the Streptococcus pyogenes derived SpCas9-NG is more limited and recognises 5′-NGN-3′ (e.g., N (A/G), NTG, GT (A/C/T), (A/G/C) CG (A/G/T) and TCG (A/G/T) G; Fujii et al. (2019) Sci. Rep., 9 (12878), doi: https://doi.org/10.1038/s41598-019-49394-5 and Kim et al. (2020) Nat. Bio. Engineering, 4:111-124, doi: https://doi.org/10.1038/s41551-019-0505-1). Cas12a and variants thereof recognize thymine-rich PAM sequences at the 5′ end of the protospacer with TTTV (where V is A, G or C) being the optimal PAM. AsCas 12a and LbCas 12a also recognize C-containing PAMs, such as CTTV, TCTV and TTCV, while CeCas 12a is more specific for the TTTV PAM (Chen et al. (2020), Genome Biol., 21,78, doi: https://doi.org/10.1186/s13059-020-01989-2). Thus, in certain embodiments the PAM sequence herein or any part thereof is any PAM sequence suitable for the RNA-guided DNA endonuclease to be used together with the gRNA libraries generated by the present method.

Reference herein to an “insertional enzyme complex” is intended to refer to a synaptic complex of an insertional enzyme and one or more tagmentation adapter polynucleotides. The insertional enzyme mediates the fragmentation of the target polynucleotide(s) to generate a plurality of cleavage fragments, after which the insertional enzyme ligates the tagmentation adapter polynucleotides at both ends of the cleavage fragment to generate a tagged cleavage fragment. The term “tagged cleavage fragment” as used herein, refers to adapter-attached polynucleotides wherein each polynucleotide is flanked by one or more tagmentation adapters. Such a system, commonly referred to as ‘tagmentation’, is described in various publications such as Adey et al (2010) Genome Biology 11: R119; Goryshin and Reznikoff (1998) The Journal of Biological Chemistry 273:7367-7374; Picelli et al (2014) Genome Research 24:2033-2040; and Caruccio (2011) Methods in Molecular Biology 733:241-255.

Reference herein to an “insertional enzyme” is intended to refer to any enzyme capable of inserting a nucleic acid sequence into a polynucleotide. In some cases, the insertional enzyme can insert the nucleic acid sequence into the polynucleotide in a sequence-independent manner. The insertional enzyme can be prokaryotic or eukaryotic. Examples of insertional enzymes include, but are not limited to, transposases, HERMES, and HIV integrase.

In some embodiments, the insertional enzyme is a transposase. Reference herein to a “transposase” is intended to refer to any enzyme with transposase activity in vitro and/or in vivo. In some embodiments, the transposase is Tn5 transposase. Other examples of appropriate transposases include, but are not limited to, Mos1, Sleeping Beauty, piggyBac, Hsmar1 and ISY100 transposases.

In further embodiments, the Tn5 transposase may be a variant Tn5 transposase which comprises one or more sequence variations compared to wild-type Tn5 transposase. In further embodiments, the variant Tn5 transposase is a hyperactive Tn5 transposase. Hyperactive Tn5 transposases comprise one or more mutations compared to wild-type Tn5 transposase which results in enzyme hyperactivity where the activity of a modified enzyme variant is considerably higher than the activity of the wild-type enzyme. Examples of mutations resulting in a hyperactive Tn5 transposase include, but are not limited to, L372P (i.e., the replacement of leucine at amino acid position 372 with proline), E54K, E110K, E345K, P242A and P242G (summarised in Reznikoff (2003) Molecular Microbiology, 47 (5): 1199-1206).

In some embodiments, the transposase may be a transposase fusion protein. Reference herein to a “fusion protein” is intended to refer to a protein consisting of at least two domains that are encoded by separate genes that have been joined so that they are transcribed and translated as a single unit, producing a single polypeptide. For example, the Cleavage Under Targets and Tagmentation (CUT&Tag) method described by Hatice et al (2019) Nature Communications 10 (1930) utilises a hyperactive Tn5 transposase—Protein A (pA-Tn5) fusion protein. In CUT&Tag, a chromatin protein is bound in situ by a target-specific antibody, which then tethers a protein A-Tn5 transposase fusion protein to ensure that the transposase only cuts the DNA at close proximity to the target chromatin protein. Other examples of appropriate transposase fusion proteins include the fusion of transposases to transcription activator-like effectors (TALE) proteins, Gal4, ZFP, Cas9 or catalytically inactive Cas9 (dCas9) (summarised in Bhatt and Chalmers (2019) Nucleic Acids Research 47 (15): 8126-8135).

Reference herein to a “tagmentation adapter” is intended to refer to any DNA oligonucleotide that comprises the nucleotide sequences required to form a functional insertional enzyme complex. For example, efficient transposition with Tn5 transposase requires that each adapter polynucleotide has a specific 19-bp transposase recognition sequence (Mosaic End or ME sequence) at each of its ends. The tagmentation adapter polynucleotide can further comprise additional sequences (e.g., restriction sites or primer sequences) as needed or desired.

In some embodiments, the tagmentation adapter additionally comprises polymerase chain reaction (PCR) handles. PCR handles are nucleotide sequences to which PCR primers bind during PCR amplification. Examples of suitable PCR handles include, but are not limited to, Nextera i7/i5 Adapters. In further embodiments, the methods disclosed herein additionally comprise PCR amplification of the tagged cleavage fragments. For any downstream ligation reactions to occur, at least one of the DNA ends to be ligated must contain a 5′ phosphate. Therefore, in some embodiments, the tagmentation adapter is phosphorylated. In some embodiments, the tagmentation adapter may comprise amino group modifications, for example, amino modifier addition, to prevent self-ligation of adapters.

In one embodiment, the method of the invention comprises the following steps:
(a) incubation of the target polynucleotide(s) with insertional enzyme complexes comprising (i) an insertional enzyme and (ii) one or more tagmentation adapters which comprise a first restriction site, to generate a plurality of adapter-attached polynucleotide fragments;
(b) amplification of the product of step (a);
(c) restriction digestion of the product of step (b) with a restriction enzyme which recognises the first restriction site and cleaves the adapter-attached polynucleotide fragment at a site downstream so as to remove the tagmentation adapters from the polynucleotide fragment;
(d) ligation of the digested product of step (c) to a plurality of first ligation adapters which comprise a second restriction site, a third restriction site, and a label, to generate a plurality of adapter-attached polynucleotide fragments wherein each polynucleotide fragment is flanked by two first ligation adapters;
(e) restriction digestion of the product of step (d) with a restriction enzyme which recognises the second restriction site and cleaves the adapter-attached polynucleotide fragments within the polynucleotide region to generate a plurality of adapter-attached polynucleotide fragments comprising a first ligation adapter, a crRNA sequence and part or all of a protospacer adjacent motif (PAM) sequence;
(f) ligation of the digested product of step (e) to a plurality of second ligation adapters which comprise a fourth restriction site and either part or all of a PAM sequence to generate a plurality of adapter-attached polynucleotide fragments wherein the polynucleotide fragment is flanked by a first ligation adapter and a second ligation adaptor;
(g) restriction digestion of the product of step (f) with a restriction enzyme which recognises the fourth restriction site and cleaves the adapter-attached polynucleotide fragment at a site downstream so as to remove the second ligation adaptor and the PAM sequence;
(h) restriction digestion of the product of step (g) with a restriction enzyme which recognises the third restriction site and cleaves the adapter-attached polynucleotide fragment at a site downstream so as to remove the first ligation adaptor, generating a plurality of gRNAs; and
(i) ligation of the digested product of step (h) to a plurality of third and fourth ligation adapters which comprise vector cloning sequences, to generate a plurality of adapter-attached gRNAs.

References herein to the term “ligation”, “ligate”, or “ligating” refers to any linkage of two nucleic acid sequences, usually comprising a phosphodiester bond. The linkage is normally facilitated by the presence of a catalytic enzyme (i.e., for example, a ligase such as T4 DNA ligase) in the presence of co-factor reagents and an energy source (i.e., for example, adenosine triphosphate (ATP)). Ligase enzymes are also available from commercial sources, such as Instant Sticky-end Ligase Master Mix and Blunt TA/Ligase Master Mix (both available from New England Biolabs).

Reference herein to a “ligation adapter” is intended to refer to any oligonucleotide suitable for ligation to another polynucleotide sequence. The ligation adapter can further comprise additional sequences (e.g., restriction sites or primer sequences) as needed or desired.

In some embodiments, the ligation adapter additionally comprises polymerase chain reaction (PCR) handles. PCR handles are nucleotide sequences to which PCR primers bind during PCR amplification. Examples of suitable PCR handles include, but are not limited to, TruSeq or Nextera i7/i5 Adapters. For any downstream ligation reactions to occur, at least one of the DNA ends to be ligated must contain a 5′ phosphate. Therefore, in some embodiments, the ligation adapter is phosphorylated. In some embodiments, the ligation adapter may comprise amino group modifications, for example, amino modifier addition, to prevent self-ligation of adapters.

Step (b) comprises amplification of the product of step (a). Reference herein to ‘amplification’ is intended to refer to any method of increasing the number of copies of an oligonucleotide sequence. Amplification is a commonly performed procedure and the skilled person would recognise that a range of techniques would be suitable to perform this step. Examples of suitable techniques include, but are not limited to, polymerase chain reaction (PCR), loop mediated isothermal amplification (LAMP), nucleic acid sequence based amplification (NASBA), self-sustained sequence replication (3SR), and rolling circle amplification (RCA).

In some embodiments, step (d) additionally comprises amplification of the adapter-attached polynucleotide fragments. In some embodiments, step (f) additionally comprises amplification of the adapter-attached polynucleotide fragments. In some embodiments, step (i) additionally comprises amplification of the adapter-attached gRNAs.

The first ligation adaptor comprises a label. The term “label” refers to a specific moiety having a unique affinity for a ligand (i.e. an affinity tag). Such a label may include, but is not limited to, a biotin label, a histidine label (i.e. 6His), or a FLAG label. In one embodiment, the label is biotin.

In some embodiments, step (a), (b), (c), (d), (e), (f), (g), (h) and/or step (i) additionally comprise a purification step. The purification step is useful to remove unwanted sequences after amplification, restriction digestion and to remove any unligated adapter sequences remaining after ligation. DNA purification is a commonly performed procedure and the skilled person would recognise that a range of techniques would be suitable to perform this step. Examples of suitable techniques include, but are not limited to, the use of magnetic beads or absorption onto a solid matrix (e.g., commercially available DNA purification columns).

In some embodiments the first restriction site of step (a) is an Mmel restriction site. In further embodiments, the restriction enzyme of step (c) which recognises the first restriction site and cleaves the adapter-attached polynucleotide fragment at a site downstream so as to remove the tagmentation adapters from the polynucleotide fragment is Mmel. Mmel is a Type IIS restriction enzyme which recognises the sequence TCCRAC, wherein R is A or G, and makes a 2 bp staggered cut 20 bases downstream. When the Mmel recognition site is placed at the correct distance (i.e., around 20 base pairs) from the junction between the tagmentation adaptor and the polynucleotide fragment, this results in the Mmel restriction enzyme cleaving the adapter-attached polynucleotide fragment at the junction between the tagmentation adaptor and the polynucleotide fragment, therefore removing the tagmentation adapters from the polynucleotide fragment. The skilled person would recognise that other restriction enzymes may be used to achieve the same result, as long as the first restriction site is placed within the tagmentation adaptor at the appropriate distance away from the junction between the tagmentation adaptor and the polynucleotide fragment. For example, NmeAlll recognises the sequence GCCGAG and makes a 2 bp staggered cut 21 bases downstream, therefore the NmeAlll restriction site would be placed around 21 base pairs away from the junction between the tagmentation adaptor and the polynucleotide fragment. Examples of other appropriate restriction enzymes include, but are not limited to, the Mmel family of restriction enzymes (e.g., ApyPI, Aqull, Aqulll, AquIV, Bsbl, Cdpl, CstMI, DraRI, DrdIV, Maql, Mmel, NhaXI, NIaCI, NmeAlll, PlaDI, PspOMII, PspPRI, Reel, RpaB5I, SdeAI, and SpoDI), EcoPI5I and NmeAll.

In some embodiments, the second restriction site of step (d) is an EcoP15I restriction site. In further embodiments, the restriction enzyme of step (e) which recognises the second restriction site and cleaves the adapter-attached polynucleotide fragments within the polynucleotide region to generate a plurality of adapter-attached polynucleotide fragments comprising a first ligation adapter, a crRNA sequence and either part of or complete PAM sequence is EcoP15I. EcoP15I recognises the sequence CAGCAG and makes a 2 bp staggered cut 25 bases downstream. Therefore, when the EcoP15I restriction site is placed at the appropriate position in the first ligation adapter (i.e., about 4 base pairs from the junction between the ligation adaptor and the polynucleotide fragment), this results in the EcoP15I restriction enzyme cleaving the adapter-attached polynucleotide fragment within the polynucleotide region to generate a plurality of adapter-attached polynucleotide fragments comprising a first ligation adapter, crRNA sequence and part of PAM sequence which is about 21 nucleotides long. The skilled person would recognise that other restriction enzymes may be used to achieve the same result, as long as the second restriction site is located within the first ligation adaptor at the appropriate distance away from the junction between the first ligation adaptor and the polynucleotide fragment. Examples of other appropriate restriction enzymes include, but are not limited to, the Mmel family of restriction enzymes (e.g., ApyPI, Aqull, Aqulll, AquIV, Bsbl, Cdpl, CstMI, DraRI, DrdIV, Maql, Mmel, NhaXI, NIaCI, NmeAlll, PlaDI, PspOMII, PspPRI, Reel, RpaB5I, SdeAI, and SpoDI), and NmeAll.

In some embodiments, the third restriction site of step (d) is an Acul restriction site. In further embodiments, the restriction enzyme of step (h) which recognises the third restriction site and cleaves the adapter-attached polynucleotide fragment at a site downstream so as to remove the first ligation adaptor is Acul. Acul recognises the sequence CTGAAG and makes a 2 bp staggered cut 16 bases downstream. Therefore, when the Acul restriction site is placed at the appropriate position in the first ligation adapter (i.e., about 15 base pairs from the junction between the ligation adaptor and the polynucleotide fragment), this results in the Acul restriction enzyme cleaving the adapter-attached polynucleotide fragment at the junction between the first ligation adaptor and the polynucleotide fragment, therefore removing the first ligation adapter from the polynucleotide fragment. The skilled person would recognise that other restriction enzymes may be used to achieve the same result, as long as the third restriction site is located within the first ligation adaptor at the appropriate distance away from the junction between the first ligation adaptor and the polynucleotide fragment.

In some embodiments, the fourth restriction site of step (f) is an Ecil restriction site. In further embodiments, the restriction enzyme of step (g) which recognises the fourth restriction site and cleaves the adapter-attached polynucleotide fragment at a site downstream so as to remove the second ligation adaptor and the PAM sequence is Ecil. Ecil recognises the sequence GGCGGA and makes a 2 bp staggered cut 11 bases downstream. Therefore, when the Ecil restriction site is placed at the appropriate position in the second ligation adapter (i.e., about 6 base pairs from the junction between the second ligation adaptor and the polynucleotide fragment), this results in the Ecil restriction enzyme cleaving the adapter-attached polynucleotide fragment at the junction between the second ligation adaptor and the polynucleotide fragment, therefore removing the second ligation adapter and the remaining PAM sequence from the polynucleotide fragment. The skilled person would recognise that other restriction enzymes may be used to achieve the same result, as long as the fourth restriction site is located within the second ligation adaptor at the appropriate distance away from the junction between the second ligation adaptor and the polynucleotide fragment.

Step (i) comprises ligation of the digested product of step (h) to a plurality of third and fourth ligation adapters which comprise vector cloning sequences, to generate a plurality of adapter-attached gRNAs. Reference herein to ‘vector cloning sequences’ is intended to refer to any sequence which allows for the cloning of an oligonucleotide into a vector. There are various techniques available for cloning into vectors, which include, but are not limited to, restriction enzyme cloning, Gateway® recombination cloning, TOPO® cloning, Gibson Assembly (Isothermal Assembly Reaction), Type IIS Assembly (e.g., Golden Gate & MoClo), and ligation independent cloning (LIC). The skilled person would understand which vector cloning sequences are appropriate depending on which technique is to be used. The third and fourth ligation adapters can further comprise additional sequences as needed or desired. For example, the adapters may include additional restriction enzyme sites, all or part of a tracrRNA sequence, all or part of a promoter sequence for expression of gRNAs from the vector, and/or sequences intended to improve the expression of gRNAs from the vector.

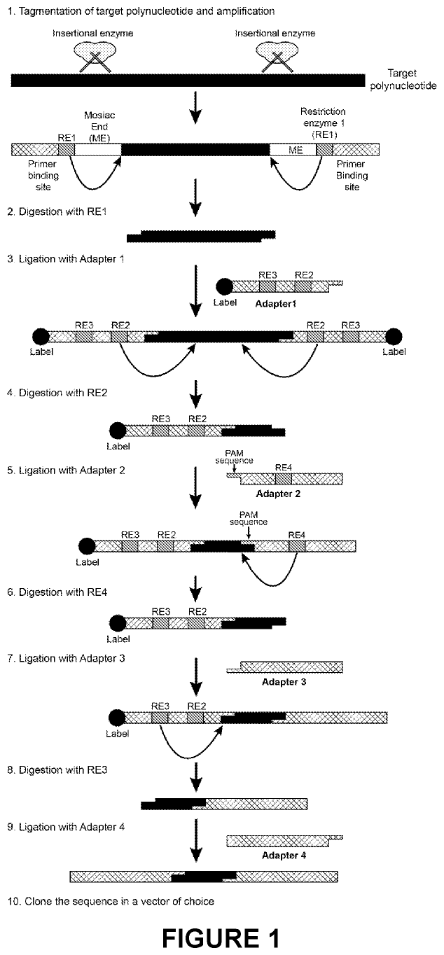

A representative, and non-limiting, method of generating a library of polynucleotide molecules encoding guide RNAs (gRNAs) is illustrated by the flowchart in FIG. 1.

According to a further aspect of the invention there is provided, a library of polynucleotide molecules encoding guide RNAs (gRNAs) obtainable by the methods as disclosed herein.

### Kits

According to a further aspect of the invention there is provided a guide RNA (gRNA) library generation kit which comprises:
(a) an insertion enzyme;
(b) a plurality of tagmentation adapters which comprise a first restriction site;
(c) a restriction enzyme which recognises the first restriction site;
(d) a plurality of first ligation adapters which comprise a second restriction site, a third restriction site, and a label;
(e) a restriction enzyme which recognises the second restriction site;
(f) a plurality of second ligation adapters which comprise a fourth restriction site and part or all of a protospacer adjacent motif (PAM) sequence;
(g) a restriction enzyme which recognises the fourth restriction site;
(h) a restriction enzyme which recognises the third restriction site; and
(i) a plurality of third and fourth ligation adapters which comprise vector cloning sequences.
In some embodiments, the kit further comprises instructions for use of the kit in accordance with any of the methods defined herein.

In one embodiment, the first restriction site is an Mmel restriction site.

In one embodiment, the restriction enzyme which recognises the first restriction site is Mmel.

In one embodiment, the second restriction site is an EcoP15I restriction site.

In one embodiment, the restriction enzyme which recognises the second restriction site is EcoP15I.

In one embodiment, the third restriction site is an Acul restriction site.

In one embodiment, the restriction enzyme which recognises the third restriction site is Acul.

In one embodiment, the fourth restriction site is an Ecil restriction site.

In one embodiment, the restriction enzyme which recognises the fourth restriction site is Ecil.

In one embodiment, the PAM sequence is 5′-NGG-3′, where N can be any nucleotide base. In another embodiment, the PAM sequence is 5′-NGN-3′. In a further embodiment, the PAM sequence is 5′-NRN-3′ or 5′-NYN-3′, where R is A or G and Y is C or T. In a yet further embodiment, the PAM sequence is NG, NNG, GAT or CAA. In a still further embodiment, the PAM sequence is 5′-NGN-3′. As hereinbefore, according to these embodiments N can be any nucleotide base.

In one embodiment, the transposase is Tn5 transposase.

The following studies and protocols illustrate embodiments of the methods described herein and their suitability for use:

### EXAMPLES

### Example 1

### Tn5 Expression

C3013 cells (NEB) were transformed with pTBX1-Tn5 plasmid (Addgene #60240; Picelli et al. (2014) Genome Research, 24 (12), 2033-2040) and plated in LB-Agar plates supplemented with ampicillin. A single colony was picked and cultured in 5 mL LB media containing ampicillin at 37° C. until the OD600 reached 0.7-0.9. Cells were chilled at 10° C. and 250 μl of 1 M IPTG was added. Cells were grown for 4 h at 23° C. Cells were pelleted at 6500 rpm (JA25.50 rotor) for 20 min. The supernatant was removed and the cells were frozen at −70° C.

### Example 2

### Oligo Preparation

All primers were ordered from IDT or Sigma.

TABLE 1
Oligos used in cus-ATAC-CRISPR experiments

| Oligo name | Sequence (5′ to 3′) |
|---|---|
| TAG-NextA- | GTCTCGTGGGCTCGGTCCGACTAGATGTGTATAAGAGACAGTTCCC |
| PCR-FP | TACCATGGTGTTCC (SEQ ID NO: 1) |
| TAG-NextB- | TCGTCGGCAGCGTCTCCGACTAGATGTGTATAAGAGACAGGTGTAG |
| PCR-RP | GTTTAGGGGCTGGT (SEQ ID NO: 2) |
| TAG-Adapt1- | GTCTCGTGGGCTCGGTCCGACTAGATGTGTATAAGAGACAG (SEQ |
| i7-FW | ID NO: 3) |
| TAG-Adapt1- | TCGTCGGCAGCGTCTCCGACTAGATGTGTATAAGAGACAG (SEQ |
| i5-FW | ID NO: 4) |
| MEDS-REV | /5Phos/CTGTCTCTTATACACATCT (SEQ ID NO: 5) |
| MEDS-B | GTCTCGTGGGCTCGGAGATGTGTATAAGAGACAG (SEQ ID NO: |
| | 6) |
| MEDS-A | TCGTCGGCAGCGTCAGATGTGTATAAGAGACAG (SEQ ID NO: |
| | 7) |
| A1-Rn1-Fw1 | /5Biosg/GACTTACTGAAGATCTACAGCAGTGAG (SEQ ID |
| | NO: 8) |
| A1-Rn1-Rw1 | /5Phos/CACTGCTGTAGATCTTCAGTAAGTC (SEQ ID NO: |
| | 9) |
| A1-Rn1-Fw2 | /5Biosg/CGATCTCTGAAGCATTGCAGCAGCGAG (SEQ ID |
| | NO: 10) |
| A1-Rn1-Rw2 | /5Phos/CGCTGCTGCAATGCTTCAGAGATCG (SEQ ID NO: |
| | 11) |
| A1-Rn1- | GTGACTGGAGTTCAGACGTGTGCTCTTCCGATCTCGATCTCTGAAG |
| PCR1_i7 | CATTGCAGCAGC (SEQ ID NO: 12) |
| A1-Rn1- | ACACTCTTTCCCTACACGACGCTCTTCCGATCTGACTTACTGAAGA |
| PCR1_15 | TCTACAGCAGTGAG (SEQ ID NO: 13) |
| A1-Rn1- | ACACTCTTTCCCTACACGACGCTCTTCCGATCTCGATCTCTGAAGC |
| Fw2_PCR1_i5 | ATTGCAGCAGC (SEQ ID NO: 14) |
| A2-Rn1-Fw1 | /5Phos/GGCTCACATCCGCCGTTGTC (SEQ ID NO: 15) |
| A2-Rn1-Rw1 | GACAACGGCGGATGTGAG (SEQ ID NO: 16) |
| A2-Rn1- | GTGACTGGAGTTCAGACGTGTGCTCTTCCGATCTGACAACGGCGGA |
| Rw1_PCR1_i7 | TGTGAG (SEQ ID NO: 17) |
| A3-Rn1-v2- | /5Phos/GTTTAAGAGCTATGCTGGAAACAGCATAGCAAGTTTAAA |
| Fw | TAAGGCTAGTCCGTTATC (SEQ ID NO: 18) |
| A3-Rn1-v2- | GATAACGGACTAGCCTTATTTAAACTTGCTATGCTGTTTCCAGCAT |
| Rw | AGCTCTTAAACNN (SEQ ID NO: 19) |
| Amplify_A3- | GTGACTGGAGTTCAGACGTGTGCTCTTCCGATCTGATAACGGACTA |
| Rn1-i7 | GCCTTATTTAAAC (SEQ ID NO: 20) |
| A4-Rn1-v2- | CTTGAAAGTATTTCGATTTCTTGGCTTTATATATCTTGTGGAAAGG |
| Fw | ACGAAACACCGN (SEQ ID NO: 21) |
| A4-Rn1-v2- | /5Phos/GGTGTTTCGTCCTTTCCACAAGATATATAAAGCCAAGAA |
| Rw | ATCGAAATACTTTCAAG (SEQ ID NO: 22) |
| Amplify_A4- | ACACTCTTTCCCTACACGACGCTCTTCCGATCTCTTGAAAGTATTT |
| Rn1-i5 | CGATTTCTTGG (SEQ ID NO: 23) |
| Chk_Bio_A1_ | /5Biosg/GACTTACTGAAGATCTACAGCAGTG (SEQ ID NO: |
| Rn1_FP | 24) |
| Chk_Bio_A1 | /5Biosg/CGATCTCTGAAGCATTGCAG (SEQ ID NO: 25) |
| Rn1_RP | |
| Nextera i701 | CAAGCAGAAGACGGCATACGAGATCGAGTAATGTCTCGTGGGCTCG |
| | G (SEQ ID NO: 26) |
| Nextera i702 | CAAGCAGAAGACGGCATACGAGATTCTCCGGAGTCTCGTGGGCTCG |
| | G (SEQ ID NO: 27) |
| Nextera i703 | CAAGCAGAAGACGGCATACGAGATAATGAGCGGTCTCGTGGGCTCG |
| | G (SEQ ID NO: 28) |
| Nextera i501 | AATGATACGGCGACCACCGAGATCTACACTATAGCCTTCGTCGGCA |
| | GCGTC (SEQ ID NO: 29) |
| Nextera i502 | AATGATACGGCGACCACCGAGATCTACACATAGAGGCTCGTCGGCA |
| | GCGTC (SEQ ID NO: 30) |
| Nextera i503 | AATGATACGGCGACCACCGAGATCTACACCCTATCCTTCGTCGGCA |
| | GCGTC (SEQ ID NO: 31) |
| Nextera i704 | CAAGCAGAAGACGGCATACGAGATGGAATCTCGTCTCGTGGGCTCG |
| | G (SEQ ID NO: 32) |
| Nextera i705 | CAAGCAGAAGACGGCATACGAGATTTCTGAATGTCTCGTGGGCTCG |
| | G (SEQ ID NO: 33) |
| Nextera i706 | CAAGCAGAAGACGGCATACGAGATACGAATTCGTCTCGTGGGCTCG |
| | G (SEQ ID NO: 34) |
| Nextera i707 | CAAGCAGAAGACGGCATACGAGATAGCTTCAGGTCTCGTGGGCTCG |
| | G (SEQ ID NO: 35) |
| NEBNext i501 | AATGATACGGCGACCACCGAGATCTACACTATAGCCTACACTCTTT |
| | CCCTACACGACGCTCTTCCGATCT (SEQ ID NO: 36) |
| NEBNext i502 | AATGATACGGCGACCACCGAGATCTACACATAGAGGCACACTCTTT |
| | CCCTACACGACGCTCTTCCGATCT (SEQ ID NO: 37) |
| NEBNext i503 | AATGATACGGCGACCACCGAGATCTACACCCTATCCTACACTCTTT |
| | CCCTACACGACGCTCTTCCGATCT (SEQ ID NO: 38) |
| NEBNext i504 | AATGATACGGCGACCACCGAGATCTACACGGCTCTGAACACTCTTT |
| | CCCTACACGACGCTCTTCCGATCT (SEQ ID NO: 39) |
| NEBNext i505 | AATGATACGGCGACCACCGAGATCTACACAGGCGAAGACACTCTTT |
| | CCCTACACGACGCTCTTCCGATCT (SEQ ID NO: 40) |
| NEBNext i506 | AATGATACGGCGACCACCGAGATCTACACTAATCTTAACACTCTTT |
| | CCCTACACGACGCTCTTCCGATCT (SEQ ID NO: 41) |
| NEBNext i701 | CAAGCAGAAGACGGCATACGAGATCGAGTAATGTGACTGGAGTTCA |
| | GACGTGTGCTCTTCCGATCT (SEQ ID NO: 42) |
| NEBNext i702 | CAAGCAGAAGACGGCATACGAGATTCTCCGGAGTGACTGGAGTTCA |
| | GACGTGTGCTCTTCCGATCT (SEQ ID NO: 43) |
| NEBNext i703 | CAAGCAGAAGACGGCATACGAGATAATGAGCGGTGACTGGAGTTCA |
| | GACGTGTGCTCTTCCGATCT (SEQ ID NO: 44) |
| NEBNext i704 | CAAGCAGAAGACGGCATACGAGATGGAATCTCGTGACTGGAGTTCA |
| | GACGTGTGCTCTTCCGATCT (SEQ ID NO: 45) |
| NEBNext i705 | CAAGCAGAAGACGGCATACGAGATTTCTGAATGTGACTGGAGTTCA |
| | GACGTGTGCTCTTCCGATCT (SEQ ID NO: 46) |
| NEBNext i706 | CAAGCAGAAGACGGCATACGAGATACGAATTCGTGACTGGAGTTCA |
| | GACGTGTGCTCTTCCGATCT (SEQ ID NO: 47) |
| NEBNext i707 | CAAGCAGAAGACGGCATACGAGATAGCTTCAGGTGACTGGAGTTCA |
| | GACGTGTGCTCTTCCGATCT (SEQ ID NO: 48) |
| NEBNext i708 | CAAGCAGAAGACGGCATACGAGATGCGCATTAGTGACTGGAGTTCA |
| | GACGTGTGCTCTTCCGATCT (SEQ ID NO: 49) |

### Example 3

### Annealing of Oligos for Tn5 Loading

The oligos TAG-Adapt1-i7-FW, TAG-Adapt1-15-FW and MEDS-REV (Table 1) were dissolved in TE buffer at a concentration of 10 nmoles/μl.

250 nmoles of TAG-Adapt1-i7-FW and MEDS-REV were mixed in a tube and heated to 95° C. for 10 min. The tube was then allowed to cool down to room temp for 1 h.

Similarly, 250 nmoles of TAG-Adapt1-i5-FW and MEDS-REV were mixed and heated to 95° C. for 10 min and subsequently allowed to cool for 1 h at room temperature.

### Example 4

### Tn5 Purification and Loading of Oligos on Chitin Magnetic Beads

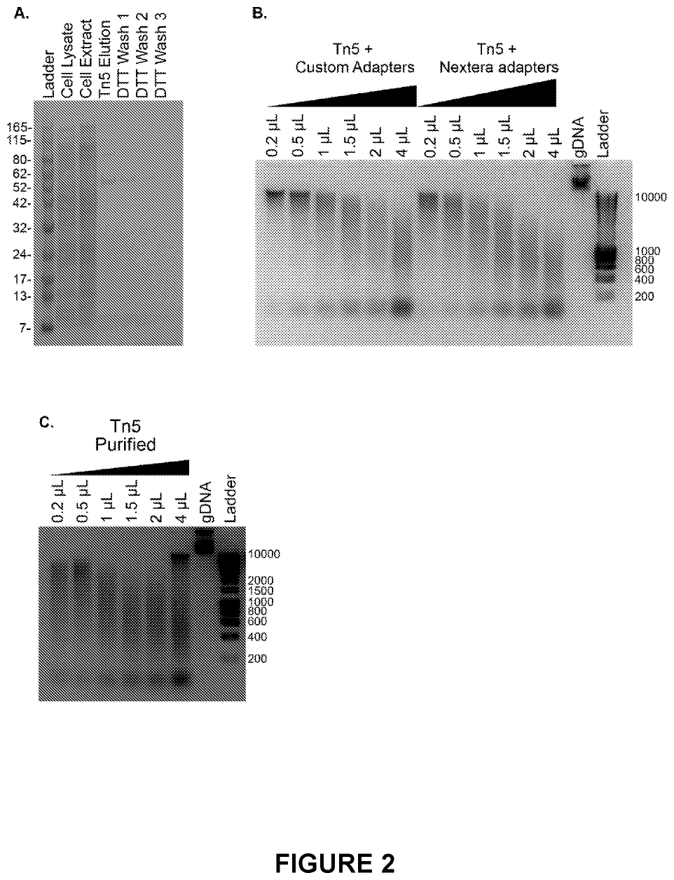

Frozen C3013 pellet expressing Tn5 (as per Example 1) was thawed and resuspended in 7 mL of cold HEGX buffer (20 mM HEPES-KOH (PH 7.2), 0.8 M NaCl, 1 mM EDTA, 10% glycerol and 0.2% Triton X-100) containing complete protease inhibitor cocktail (PIC). The cells were sonicated on 80% power with Bioruptor sonicator for eight times with 30 s on and 30 s off intervals on ice. The lysate was centrifuged at 11000 rpm for 30 min at 4° C. The supernatant was then transferred to a new beaker and 2.1 mL of 10% neutralised polyethyleneimine (PEI) was added in a dropwise manner with regular stirring to precipitate DNA. The solution was then centrifuged at 11000 rpm for 20 min at 4° C. The supernatant was transferred to a new tube and 100 μl of this sample was set aside as a cell lysate control (FIG. 2A).

In order to prepare the chitin magnetic resin (NEB), 2 mL of the chitin resin was washed with 10 mL HEGX buffer twice. The clarified supernatant (7 mL) from before was then added to the chitin resin. The tube was rotated for 30 min at 4° C. and subsequently placed on a magnetic column to remove the supernatant. 100 μl of the supernatant was kept as a control (FIG. 2A: Cell extract control). The chitin beads were washed 4 times with 10 mL cold HEGX buffer.

The chitin beads were then loaded with 250 nmoles of TAG-Adapt1-i7-FW/MEDS-REV and TAG-Adapt1-i5-FW/MEDS-REV oligos (as per Example 3) in 3 mL HEGX buffer containing PIC. The beads were rotated overnight at room temperature. The next day, chitin beads were washed 4 times with 10 mL HEGX buffer. The chitin beads were then resuspended in 5 mL HEGX buffer containing 50 mM 1,4-Dithiothreitol (DTT). The beads were incubated at 4° C. for 48 h. Subsequently, the beads were placed in a magnetic column and the supernatant containing purified Tn5 was collected. The beads were then washed three times in 1 mL HEGX buffer containing 50 mM DTT and the remaining Tn5 in the eluate was collected. All the Tn5 samples were run on 10% SDS-PAGE gel and Coomassie staining was used to detect the presence of 55 kD Tn5 band (FIG. 2A).

All Tn5 transposase samples were pooled and concentrated to 0.5 mL using 10 kD MW Vivaspin concentrator column by spinning at 4500×g. The concentrated Tn5 sample was dialysed overnight at 4° C. in 2 L of 2× Tn5 dialysis buffer (100 mM HEPES-KOH (pH 7.2), 0.2 M NaCl, 0.2 mM EDTA, 0.2% Triton X-100, 20% glycerol and 2 mM DTT) using Spectra/por 6-8 kD dialysis membrane. The dialysed sample was collected and the concentration was measured using Bradford assay. The sample was diluted to 24 μM using 2× Tn5 dialysis buffer. An equal amount of 100% glycerol was added to the Tn5 and 25μl aliquots were stored at −70° C.

### Example 5

### Transposome Assembly in Solution

The oligos MEDS-A, MEDS-B, MEDS-REV, TAG-Adapt1-i5-FW and TAG-Adapt1-i7-FW (Table 1) were dissolved in T4 DNA ligase buffer at 100 μM. 3 μL each of the following combinations of MEDS-A/MEDS-REV, MEDS-B/MEDS-REV, TAG-Adapt1-i7-FW/MEDS-REV and TAG-Adapt1-i5-FW/MEDS-REV were placed in PCR tube. The samples were heated to 95° C. for 10 min and then placed on a bench for 1 h to cool down to room temperature.

Commercially available Tn5 (7 μL) was loaded with either 1 μL of MEDS-A/MEDS-REV and MEDS-B/MEDS-REV (referred hereafter as standard Nextera adapters) or TAG-Adapt1-i7-FW/MEDS-REV and TAG-Adapt1-i5-FW/MEDS-REV (referred hereafter as custom adapters) for 1 h at room temperature to have the final transposome.

### Example 6

### Tagmentation Reaction

Assembled transposomes either in solution (as per Example 5) or on magnetic beads (as per Example 4) were used for tagmenting genomic DNA (400 ng) in 5× TAPS-DMF buffer (50 mM TAPS-NaoH, 25 mM MgCl2 and 50% DMF) in 20 μL reaction. The tagmentation reaction was carried out at 55° C. for 30 min. After the tagmentation, proteinase K (0.5 μL) was added to the reaction for 7 min at 55° C. The tagmentation reaction (5 μL) was checked on 2% agarose gel (FIGS. 2B and 2C). Tn5 was able to tagment genomic DNA (gDNA) when loaded with either standard Nextera adapters or custom adapters (FIG. 2B). Both Tn5 transposome assembled in solution (FIG. 2B) or on magnetic beads (FIG. 2C) was able to tagment the gDNA with increasing volumes of Tn5 (0.2-4 μL). The remaining tagmentation reaction (15 μL) was then purified using QIAquick PCR purification kit.

The purified products were then PCR amplified using the conditions in Table 2. The PCR primers used were either Nextera i501/i701, Nextera i502/i702 or Nextera i503/i703 (Table 1). KAPA HiFi HotStart ReadyMix was used for the PCR. The PCR products were purified using ChargeSwitch PCR clean-up beads.

TABLE 2
PCR conditions for Tagmentation Amplification

| Step | | Temperature | Time | |
|---|---|---|---|---|
| Initial Denaturation | | 95° C. | 3 | min |
| 10 cycles | Denaturation | 98° C. | 20 | s |
| | Annealing | 57° C. | 15 | s |
| | Extension | 72° C. | 1 | min |

| | | | |
|---|---|---|---|
| Final Extension | 72° C. | 2 | min |

### Example 7

### Preparation of Adapters Used for Ligations

The forward and reverse oligos were dissolved in TE buffer at final concentration of 100 μM. 10 μL of both oligos were mixed in a PCR tube and 2.2 μL of T4 DNA ligase buffer (NEB) was added. The oligos were heated to 95° C. for 10 min in PCR machine and then allowed to cool down to room temperature for 1 h. Subsequently, the oligos were diluted to 10 μM by addition of TE buffer. The adapter names and the oligos used to prepare them are listed Table 3, oligo and adapter sequences can be found in Table 1.

TABLE 3
Oligos used to prepare adapters for ligation

| Adapter name | Forward oligo | Reverse oligo |
|---|---|---|
| Adapter 1-1 | A1-Rn1-Fw1 | A1-Rn1-Rw1 |
| Adapter 1-2 | A1-Rn1-Fw2 | A1-Rn1-Rw2 |
| Adapter 2 | A2-Rn1-Fw1 | A2-Rn1-Rw1 |
| Adapter 3 | A3-Rn1-v2-Fw | A3-Rn1-v2-Rw |
| Adapter 4 | A4-Rn1-v2-Fw | A4-Rn1-v2-Rw |

### Example 8

### Defined Template to Test the Enzymatic Reactions

A defined template of known sequence was used to test enzymatic reactions. The sequence of the template is as follows:

(SEQ ID NO: 50)
5′-TCCCTACCATGGTGTTCCCCTTCGGCCAGATCTCTCAGGCCTCTGCT
CTGGCTCCAGCCCCTCCTCAGGTGCTGCCTCAGGCTCCTGCTCCTGCACC
AGCTCCAGCCATGGTGTCTGCACTGGCTCAGGCACCAGCACCCGTGCCTG
TGCTGGCTCCTGGACCTCCACAGGCTGTGGCCCCACCAGCCCCTAAACCT
ACA-3′.

Two PCR primers TAG-NextA-PCR-FP and TAG-NextB-PCR-RP (Table 1) were used to add a Mmel restriction enzyme site and Nextera A/Nextera B PCR amplification sites. KAPA HiFi HotStart ReadyMix was used for PCR using the conditions in Table 4.

TABLE 4
PCR conditions for preparation of the Defined
Template containing Mmel restriction sites

| Step | | Temperature | Time | |
|---|---|---|---|---|
| Initial Denaturation | | 95° C. | 3 | min |
| 20 cycles | Denaturation | 98° C. | 20 | s |
| | Annealing | 57° C. | 15 | s |
| | Extension | 72° C. | 15 | s |

| | | | |
|---|---|---|---|
| Final Extension | 72° C. | 1 | min |

The PCR products were purified using ChargeSwitch PCR clean-up beads.

### Example 9

### Mmel Restriction Digestion & Ligation of Adapter 1

2.5 μg of the defined template containing Mmel restriction sites (as per Example 8) or PCR amplified tagmented gDNA (from Example 6) were digested with Mmel in a 50 μL reaction at 37° C. for 1 h. The restriction digestion reaction was then purified using ZYMO DNA Clean & Concentrator kit. Mmel digested product (240 ng) was ligated with 3 μl each of biotinylated Adapter1-1 and Adapter1-2 (see Example 7) using Blunt TA/ligase mastermix (NEB) for 20 min at 25° C. The ligation products were purified using ZYMO DNA Clean & Concentrator kit.

### Example 10

### PCR Amplification of Adapter 1 Ligation Products

The Adapter 1 ligation products (from Example 9) were then PCR amplified using Chk_Bio_A1_Rn1_FP and Chk_Bio_A1_Rn1_RP primers. The PCR conditions in Table 5 and Table 6 were used with KAPA HiFi HotStart polymerase.

TABLE 5
PCR conditions for amplification of Adapter 1
ligation products for gDNA

| Step | | Temperature | Time | |
|---|---|---|---|---|
| Initial Denaturation | | 95° C. | 3 | min |
| 11 cycles | Denaturation | 98° C. | 20 | s |
| | Annealing | 57° C. | 15 | s |
| | Extension | 72° C. | 60 | s |

| | | | |
|---|---|---|---|
| Final Extension | 72° C. | 2 | min |

TABLE 6
PCR conditions for amplification of Adapter 1
ligation products for Defined Template

| Step | | Temperature | Time | |
|---|---|---|---|---|
| Initial Denaturation | | 95° C. | 3 | min |
| 11 cycles | Denaturation | 98° C. | 20 | s |
| | Annealing | 57° C. | 15 | s |
| | Extension | 72° C. | 15 | s |

| | | | |
|---|---|---|---|
| Final Extension | 72° C. | 1 | min |

ChargeSwitch PCR clean-up beads were used for purification of PCR products.

### Example 11

### Addition of Multiplexing Indexes for Next Generation Sequencing (NGS) to Adapter 1 Ligated Products

The ligation products from Example 9 or PCR amplified ligation products from Example 10 were then amplified using A1-Rn1-PCR1-i5 and A1-Rn1-PCR1-i7 primers to add PCR handles for Illumina sequencing. The PCR1 conditions in Table 7 were used with KAPA HiFi HotStart polymerase.

TABLE 7
PCR1 conditions for addition of sequencing
PCR handles to Adapter 1 ligation products

| Step | | Temperature | Time | |
|---|---|---|---|---|
| Initial Denaturation | | 95° C. | 3 | min |
| 11 cycles | Denaturation | 98° C. | 20 | s |
| | Annealing | 57° C. | 15 | s |
| | Extension | 72° C. | 60 | s |

| | | | |
|---|---|---|---|
| Final Extension | 72° C. | 2 | min |

These PCR1 products were purified using ChargeSwitch PCR clean-up beads.

The purified PCR1 products (30 ng) were used for different PCR cycles (5, 8, 10 and 12 cycles) to add P5/P7 Illumina sequencing sites and multiplexing indexes using KAPA HiFi HotStart polymerase. NEBNext i501 primer and NEBNext i701 primer were used for amplification of gDNA. NEBNext i504 Primer and NEBNext i701 Primer were used for amplification of defined template. The PCR2 conditions used were as shown in Table 8.

TABLE 8
PCR2 conditions for addition of multiplexing
indexes to Adapter 1 ligation products

| Step | | Temperature | Time | |
|---|---|---|---|---|
| Initial Denaturation | | 95° C. | 3 | min |
| 5, 8, 10 or | Denaturation | 98° C. | 20 | s |
| 12 cycles | Annealing | 65° C. | 15 | s |
| | Extension | 72° C. | 45 | s |

| | | | |
|---|---|---|---|
| Final Extension | 72° C. | 3 | min |

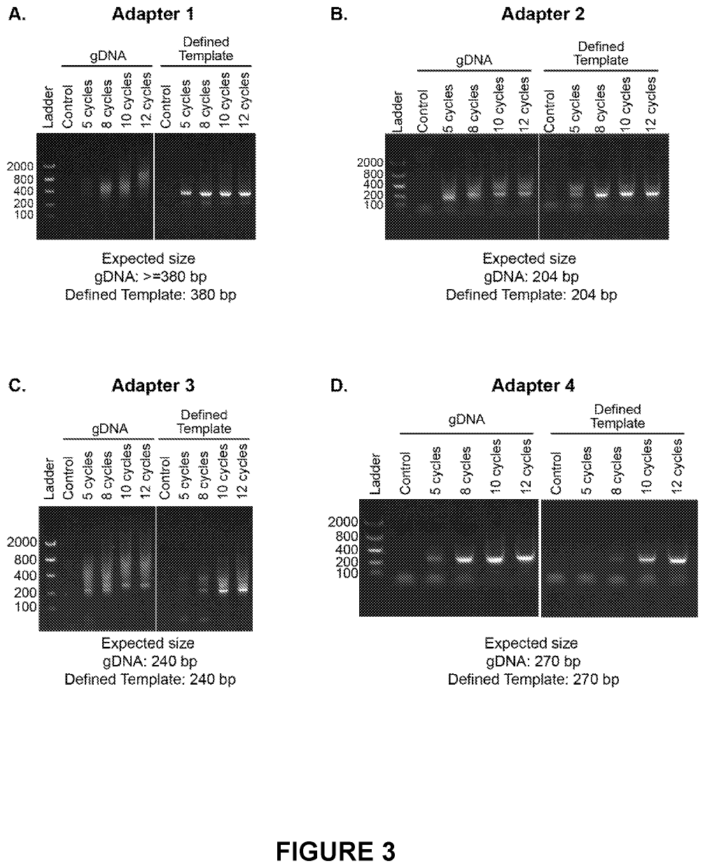

The PCR2 products were checked on 2% agarose gel for presence of the correct band (FIG. 3A) and were purified with ChargeSwitch PCR clean-up beads. The expected size for gDNA was >=380 bp and for the defined template was 380 bp (FIG. 3A).

### Example 12

### Streptavidin Bead Preparation & Biotin Adapter Loading

Binding and Washing (B&W) buffer—2× (10 mM Tris-HCl (pH 7.5), 1 mM EDTA and 2 M NaCl) was diluted to 1× with nuclease free water. Dynabeads M-270 Streptavidin beads (75 μL/sample) were washed with 1× B&W buffer (1mL) three times. Dynabeads were then dissolved in 100 μL 2× B&W buffer. Biotinylated Adapter 1 ligation products (450 ng) from Example 9 or Example 10 for gDNA and defined template were added to the dynabeads for a total volume of 200 μL. The samples were then incubated for 20 min at room temperature with shaking. The samples were then placed in DynaMag-PCR Magnet and washed thrice with 1× B&W buffer. After the final wash, the beads were resuspended in 36 μL water.

### Example 13

### EcoP15I Restriction Digestion & Ligation of Adapter 2

EcoP15I digestion was carried out directly on beads in Cutsmart buffer (NEB) supplemented with 100 μM sinefungin in a 50 μL reaction at 37° C. for 1 h. After the digestion, the beads were washed twice with 1× B&W buffer (containing 0.01% Tween-20) and three times with 1× B&W buffer. After washings, the beads were dissolved in 30 μL water.

The beads were then used for ligation with 6 μl of Adapter2 (10 μM) that contained TruSeqA/B PCR handles using Blunt TA/Ligase Master Mix. The ligation was carried out at 25° C. for 20 min. After the ligation, the beads were washed three times with 1× B&W buffer & subsequently dissolved in 40 μL water.

### Example 14

### Addition of Multiplexing Indexes to Adapter 2 Ligation Products for NGS

The EcoP15I digestion products ligated with Adapter 2 (5 μL beads) were then used for PCR amplification with A1-Rn1-PCR1_i5, A1-Rn1-Fw2_PCR1_i5 and A2-Rn1-Rw1_PCR1_i7 primers for 12 cycles with KAPA HiFi HotStart polymerase. The PCR1 parameters are shown in Table 9.

TABLE 9
PCR1 conditions for addition of sequencing
PCR handles to Adapter 2 ligation products

| Step | | Temperature | Time | |
|---|---|---|---|---|
| Initial Denaturation | | 95° C. | 3 | min |
| 12 cycles | Denaturation | 98° C. | 20 | s |
| | Annealing | 58° C. | 15 | s |
| | Extension | 72° C. | 15 | s |

| | | | |
|---|---|---|---|
| Final Extension | 72° C. | 1 | min |

The PCR1 products were then purified using ChargeSwitch PCR clean-up beads. These purified PCR 1 products (30 ng) were used for the addition of multiplexing indexes and different PCR cycles were tested (5, 8, 10 or 12 cycles) with KAPA HiFi HotStart polymerase. For gDNA, NEBNext i501 primer and NEBNext i703 primer were used while for defined template NEBNext i504 primer and NEBNext i703 primer were used. The PCR2 parameters are mentioned in Table 10. ChargeSwitch PCR clean-up beads were used for PCR2 purification.

TABLE 10
PCR2 conditions for addition of multiplexing
indexes to Adapter 2 ligation products

| Step | | Temperature | Time | |
|---|---|---|---|---|
| Initial Denaturation | | 95° C. | 3 | min |
| 5, 8, 10 or | Denaturation | 98° C. | 20 | s |
| 12 cycles | Annealing | 65° C. | 15 | s |
| | Extension | 72° C. | 45 | s |

| | | | |
|---|---|---|---|
| Final Extension | 72° C. | 3 | min |

The PCR2 products were run on 2% agarose gel. The expected band of 204 bp was observed for Adapter 2 ligated products for both gDNA and defined template (FIG. 3B).

### Example 15

### Ecil Restriction Digestion & Ligation of Adapter 3

The magnetic beads with adapter 2 ligated products from Example 13 (both gDNA and defined template) were then digested with Ecil for 1 h at 37° C. in Cutsmart buffer (NEB). After Ecil digestion, the beads were washed thrice with 1× B&W buffer and dissolved in 15 μL water. Ligation of 6 μL of Adapter 3 (10 μM) was carried out with Blunt TA/Ligase Master Mix (NEB) for 20 min at 25° C. After the ligation, the beads were washed thrice with 1× B&W buffer and then dissolved in 40 μL water.

### Example 16

### Addition of Multiplexing Indexes to Adapter 3 Ligation Products for NGS

Adapter 3 ligated beads (5 μL) were used for PCR amplification with A1-Rn1-PCR1_i5, A1-Rn1-Fw2_PCR1_i5 and Amplify_A3-Rn1-i7 primers for addition of TruSeqA/B PCR handles.

KAPA HiFi Taq polymerase was used for PCR1 with the conditions in Table 11:

TABLE 11
PCR1 conditions for addition of sequencing
PCR handles to Adapter 3 ligation products

| Step | | Temperature | Time | |
|---|---|---|---|---|
| Initial Denaturation | | 95° C. | 3 | min |
| 13 cycles | Denaturation | 98° C. | 20 | s |
| | Annealing | 57° C. | 15 | s |
| | Extension | 72° C. | 15 | s |

| | | | |
|---|---|---|---|
| Final Extension | 72° C. | 1 | min |

The PCR1 products were purified with ChargeSwitch PCR clean-up beads. These PCR1 products (30 ng) were used for addition of multiplexing indexes and either 5, 8, 10 or 12 PCR cycles were tested. For defined template sample, NEBNext i504 Primer and NEBNext i705 Primer were used whereas for gDNA sample, NEBNext i501 Primer and NEBNext i705 Primer were used. The PCR2 conditions are mentioned in Table 12. PCR2 purification was carried out using ChargeSwitch PCR clean-up beads.

TABLE 12
PCR2 conditions for addition of multiplexing
indexes to Adapter 3 ligation products

| Step | | Temperature | Time | |
|---|---|---|---|---|
| Initial Denaturation | | 95° C. | 3 | min |
| 5, 8, 10 or | Denaturation | 98° C. | 20 | s |
| 12 cycles | Annealing | 65° C. | 15 | s |
| | Extension | 72° C. | 45 | s |

| | | | |
|---|---|---|---|
| Final Extension | 72° C. | 3 | min |

The PCR2 products were run 2% agarose gel and the expected band of 240 bp could be observed (FIG. 3C). Some additional bands/smear, in addition to the correct one, were also observed probably due to PCR amplification bias.

### Example 17

### Acul Restriction Digestion & Ligation of Adapter 4

The beads containing Adapter 3 ligated products (from Example 15) were digested with Acul (NEB) in Cutsmart buffer (NEB) for 1 h at 37° C. in a 30 μL reaction. After the digestion, the enzymatic reaction containing the beads was placed on DynaMag-PCR magnet and the supernatant was transferred to a new tube. The supernatant was then supplemented with 2× Blunt TA/Ligase Master Mix and 6 μL of Adapter 4 (10 μM). The ligation was carried out at 25° C. for 20 min. ZYMO DNA Clean & Concentrator kit was used for purification of the ligation reaction.

### Example 18

### Addition of Multiplexing Indexes to Adapter 4 Ligation Products for NGS

The purified ligation products (from Example 17) were then PCR amplified using Amplify_A4-Rn1-i5 and Amplify_A3-Rn1-i7 primers using the PCR1 conditions in Table 13.

TABLE 13
PCR1 conditions for amplification of Adapter 4 ligation products

| Step | | Temperature | Time |
|---|---|---|---|
| Initial Denaturation | | 95° C. | 3 min |
| 14 cycles | Denaturation | 98° C. | 20 s |
| | Annealing | 57° C. | 15 s |
| | Extension | 72° C. | 15 s |

| | | |
|---|---|---|
| Final Extension | 72° C. | 1 min |

PCR1 products were purified with ChargeSwitch PCR clean-up beads and 30 ng of them were used for addition of multiplexing indexes using the PCR2 conditions in Table 14. For gDNA sample, NEBNext i501 Primer and NEBNext i707 Primer were used whereas for defined template sample, NEBNext i504 Primer and NEBNext i707 Primer were used for PCR amplification (5, 8, 10 or 12 cycles). The PCR2 products were purified with ChargeSwitch PCR clean-up beads.

TABLE 14
PCR2 conditions for addition of multiplexing indexes to
Adapter 4 ligation products

| Step | | Temperature | Time |
|---|---|---|---|
| Initial Denaturation | | 95° C. | 3 min |
| 5, 8, 10 or | Denaturation | 98° C. | 20 s |
| 12 cycles | Annealing | 65° C. | 15 s |
| | Extension | 72° C. | 45 s |

| | | |
|---|---|---|
| Final Extension | 72° C. | 3 min |

These PCR2 products were then run on 2% agarose gel and expected band of 270 bp was observed for both defined template and gDNA (FIG. 3D).

### Example 19

### Data Analysis of NGS

Samples in each step were individually barcoded and pooled together for next generation sequencing analysis. Miseq v2 150 bp paired kit was used to sequence the NGS library. Samples were quantified and loaded on MiSeq machine. FASTQ files were analyzed with customized scripts. Reads with expected library structure were isolated and mapped to human hg38 genome.

### Example 20

### Cell Culture and Cell Viability

Leukaemia cell line K562 were cultured in RPMI 1640 medium and 10% fetal bovine serum (FBS) at 37° C. and 5% CO₂. Cell number and viability of K562 cells were determined using Countess II automated cell counter by mixing 1:1 solution of K562 cells with 0.4% Trypan blue stain. If the samples had 5-15% dead cells, the suspension of K562 cells was treated with DNase (Worthington) at a final concentration of 200 U/ml in cell culture medium for 30 min at 37° C. and 5% CO₂. The cells were then washed with Phosphate buffered saline (PBS) and resuspended in PBS. K562 cells number and viability was determined again using Countess II.

### Example 21

### Preparation of ATAC Library

The following buffers were prepared for ATAC reaction: ATAC-resuspension buffer (ATAC-RSB) contained 10 mM Tris-HCl (pH 7.4), 10 mM NaCl and 3 mM MgCl₂. Tagmentation buffer (2×) contained 20 mM Tris-HCl (pH 7.6), 10 mM MgCl₂ and 20% dimethyl formamide. Transposition mix contained 25 μL tagmentation buffer (2×), 16.5 μL PBS, 6-8 μL transposase (12 μM stock), 0.5 μL 1% digitonin and 0.5 μL 10% Tween-20.

Either 50,000 or 100,000 K562 cells were pelleted at 500 RCF for 5 min at 4° C. in a 1.5 mL tube. The supernatant was discarded and the cell pellet was resuspended in 50 μL cold ATAC-RSB supplemented with 0.1% Tween-20, 0.1% NP40 and 0.01% Digitonin. The cells were incubated on ice for 3 minutes. 1 mL of cold ATAC-RSB containing 0.1% Tween-20 was added to the cells and the tube was inverted 3 times for mixing the contents. The nuclei were pelleted in a fixed angle centrifuge at 500 RCF for 10 min (4° C.). The supernatant was discarded and the pellet is resuspended in 50 μL of transposition mix at 37° C. for 30 min in a thermomixer at 300 RPM.

The transposition reaction was then cleaned with Zymo DNA clean and concentrator-5 columns and eluted in 21 μL elution buffer. All of the eluate was used for amplification with the PCR conditions mentioned in Table 15 using NEBNext 2×MasterMix. The primers used for amplification were i7-NexteraA (GTCTCGTGGGCTCGGTC; SEQ ID NO: 51) and i5-NexteraB (TCGTCGGCAGCGTCTC; SEQ ID NO: 52).

TABLE 15
PCR conditions for amplification of tagmented ATAC sample

| Step | | Temperature | Time |
|---|---|---|---|
| Gap filling | | 72° C. | 5 min |
| Initial Denaturation | | 98° C. | 30 s |
| 10 cycles | Denaturation | 98° C. | 10 s |
| | Annealing | 61° C. | 30 s |
| | Extension | 72° C. | 90 s |

| | | |
|---|---|---|
| Final Extension | 72° C. | 3 min |

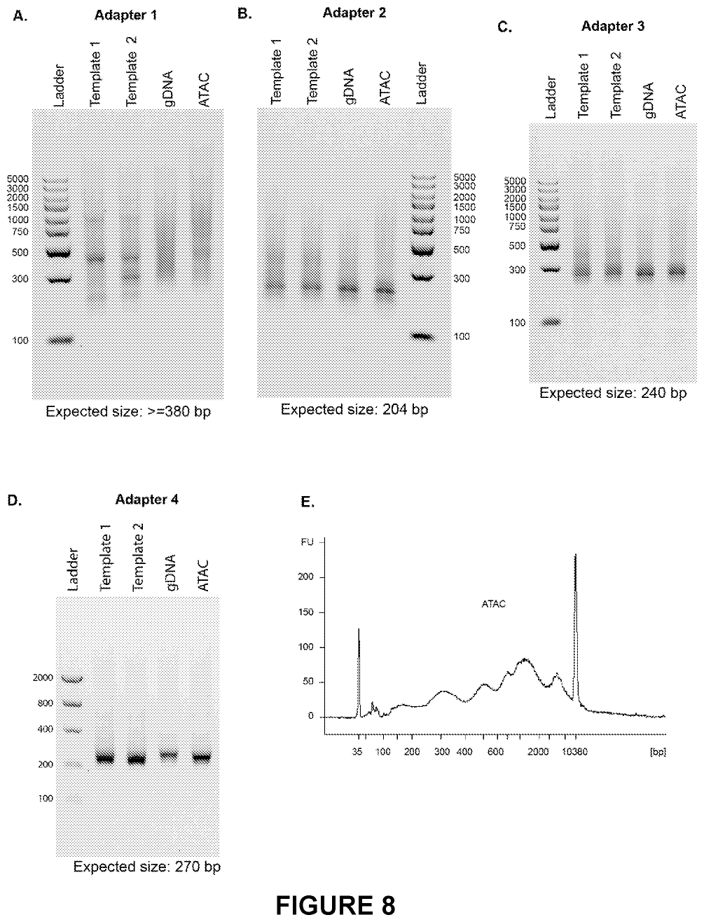

The PCR products were purified using Zymo DNA clean and concentrator-5 columns. The purified PCR products were analysed on Bioanalyzer using Agilent high sensitivity DNA kit (FIG. 8E).

### Example 22

### Tagmentation of Genomic DNA (gDNA)

Assembled transposomes on magnetic beads (as per Example 4) were used for tagmenting genomic DNA (400 ng) from K562 cells in 5× TAPS-DMF buffer (50 mM TAPS-NaoH, 25 mM MgCl2 and 50% DMF) in 20 μL reaction. The tagmentation reaction was carried out at 55° C. for 30 min. After the tagmentation, proteinase K (0.5 μL) was added to the reaction for 7 min at 55° C. The tagmentation reaction was then purified using Zymo DNA clean and concentrator-5 kit.

The purified products were then PCR amplified using the conditions in Table 16. The PCR primers used were i7-NexteraA and and i5-NexteraB. KAPA HiFi HotStart ReadyMix was used for the PCR. The PCR products were purified using ChargeSwitch PCR clean-up beads.

TABLE 16
PCR conditions for amplification of tagmented gDNA

| Step | | Temperature | Time |
|---|---|---|---|
| Initial Denaturation | | 95° C. | 3 min |
| 10 cycles | Denaturation | 98° C. | 20 s |
| | Annealing | 57° C. | 15 s |
| | Extension | 72° C. | 1 min |

| | | |
|---|---|---|
| Final Extension | 72° C. | 2 min |

### Example 23

### Defined Templates to Test the Enzymatic Reactions

Two defined templates of known sequence were used to test enzymatic reactions.

The sequence of the template 1 is as follows:

(SEQ ID NO: 50)
5′-TCCCTACCATGGTGTTCCCCTTCGGCCAGATCTCTCAGGCCTCTGCT
CTGGCTCCAGCCCCTCCTCAGGTGCTGCCTCAGGCTCCTGCTCCTGCACC
AGCTCCAGCCATGGTGTCTGCACTGGCTCAGGCACCAGCACCCGTGCCTG
TGCTGGCTCCTGGACCTCCACAGGCTGTGGCCCCACCAGCCCCTAAACCT
ACA-3′.

The sequence of template 2 is as follows:

(SEQ ID NO: 53)
5′-TCCCTACCATGGTGTTCCCCGGCGGCCAGATCTCTCAGGCCTCTGCT
CTGGCTCCAGCCCCTCCTCAGGTGCTGCCTCAGGCTCCTGCTCCTGCACC
AGCTCCAGCCATGGTGTCTGCACTGGCTCAGGCACCAGCACCCGTGCCTG
TGCTGGCTCCTGGACCTCCACAGGCTGTGGCCCCACCAGCCCCTAAACCT
ACA-3′.

Two PCR primers TAG-NextA-PCR-FP and TAG-NextB-PCR-RP (Table 1) were used to add a Mmel restriction enzyme site and Nextera A/Nextera B PCR amplification sites.

KAPA HiFi HotStart ReadyMix was used for PCR using the conditions in Table 17.

TABLE 17
PCR conditions for preparation of the Defined Template
containing Mmel restriction sites

| Step | | Temperature | Time |
|---|---|---|---|
| Initial Denaturation | | 95° C. | 3 min |
| 20 cycles | Denaturation | 98° C. | 20 s |
| | Annealing | 57° C. | 15 s |
| | Extension | 72° C. | 15 s |

| | | |
|---|---|---|
| Final Extension | 72° C. | 1 min |

The PCR products were purified using ChargeSwitch PCR clean-up beads.

Example 24-Mmel restriction digestion & ligation of Adapter 1 2.5 μg of the defined templates containing Mmel restriction sites (as per Example 23) or PCR amplified tagmented gDNA (from Example 22) or PCR amplified ATAC sample (from Example 21) were digested with Mmel in a 50 μL reaction at 37° C. for 1 h. The restriction digestion reaction was then purified using ZYMO DNA Clean & Concentrator kit. Mmel digested product (240 ng) was ligated with 3 μL each of biotinylated Adapter1-1 and Adapter1-2 (see Example 7) using Blunt TA/ligase mastermix (NEB) for 20 min at 25° C. The ligation products were purified using ZYMO DNA Clean & Concentrator kit.

### Example 25

### PCR Amplification of Adapter 1 Ligation Products

The Adapter 1 ligation products (from Example 24) were then PCR amplified using Chk_Bio_A1_Rn1_FP and Chk_Bio_A1_Rn1_RP primers. The PCR conditions in Table 18 and Table 19 were used with KAPA HiFi HotStart polymerase.

TABLE 18
PCR conditions for amplification of Adapter 1 ligation
products for gDNA and ATAC

| Step | | Temperature | Time |
|---|---|---|---|
| Initial Denaturation | | 95° C. | 3 min |
| 11 cycles | Denaturation | 98° C. | 20 s |
| | Annealing | 57° C. | 15 s |
| | Extension | 72° C. | 60 s |

| | | |
|---|---|---|
| Final Extension | 72° C. | 2 min |

TABLE 19
PCR conditions for amplification of Adapter1 ligation
products for Defined Template 1 and Defined Template 2

| Step | | Temperature | Time |
|---|---|---|---|
| Initial Denaturation | | 95° C. | 3 min |
| 11 cycles | Denaturation | 98° C. | 20 s |
| | Annealing | 57° C. | 15 s |
| | Extension | 72° C. | 15 s |

| | | |
|---|---|---|
| Final Extension | 72° C. | 1 min |

ChargeSwitch PCR clean-up beads were used for purification of PCR products.

### Example 26

### Addition of Multiplexing Indexes for Next Generation Sequencing (NGS) to Adapter 1 Ligated Products

The ligation products from Example 24 or PCR amplified ligation products from Example 25 were then amplified using A1-Rn1-PCR1-i5 and A1-Rn1-PCR1-i7 primers to add PCR handles for Illumina sequencing. The PCR1 conditions in Table 20 were used with KAPA HiFi HotStart polymerase.

TABLE 20
PCR1 conditions for addition of sequencing PCR handles to
Adapter 1 ligation products

| Step | | Temperature | Time |
|---|---|---|---|
| Initial Denaturation | | 95° C. | 3 min |
| 11 cycles | Denaturation | 98° C. | 20 s |
| | Annealing | 57° C. | 15 s |
| | Extension | 72° C. | 60 s |

| | | |
|---|---|---|
| Final Extension | 72° C. | 2 min |

These PCR1 products were purified using ChargeSwitch PCR clean-up beads.

The purified PCR1 products (50-100 ng) were used for 5 PCR cycles to add P5/P7 Illumina sequencing sites and multiplexing indexes using KAPA HiFi HotStart polymerase. Different combinations of NEBNext i501-1506 primers and NEBNext i701-i708 primers were used for amplification of gDNA, ATAC and defined template 1 and defined template 2. The PCR2 conditions used were as shown in Table 21.

TABLE 21
PCR2 conditions for addition of multiplexing indexes to
Adapter 1 ligation products

| Step | | Temperature | Time |
|---|---|---|---|
| Initial Denaturation | | 95° C. | 3 min |
| 5 cycles | Denaturation | 98° C. | 20 s |
| | Annealing | 65° C. | 15 s |
| | Extension | 72° C. | 45 s |

| | | |
|---|---|---|
| Final Extension | 72° C. | 3 min |

The PCR2 products were checked on 2% agarose gel for presence of the correct band (FIG. 8A) and were purified with ChargeSwitch PCR clean-up beads. The expected size for gDNA and ATAC was >=380 bp and for the defined template was 380 bp (FIG. 8A).

### Example 27

### Streptavidin Bead Preparation & Biotin Adapter Loading

Binding and Washing (B&W) buffer-2× (10 mM Tris-HCl (pH 7.5), 1 mM EDTA and 2 M NaCl) was diluted to 1× with nuclease free water. Dynabeads M-270 Streptavidin beads (75 μL/sample) were washed with 1× B&W buffer (1 mL) three times. Dynabeads were then dissolved in 100 μL 2× B&W buffer. Biotinylated Adapter 1 products (450 ng) from Example 25 for gDNA, ATAC, defined template 1 and defined template 2 were added to the dynabeads for a total volume of 200 μL. The samples were then incubated for 20 min at room temperature with shaking. The samples were then placed in DynaMag-PCR Magnet and washed thrice with 1× B&W buffer. After the final wash, the beads were resuspended in 36 μL water.

### Example 28

### EcoP15I Restriction Digestion & Ligation of Adapter 2

EcoP15I digestion was carried out directly on beads in Cutsmart buffer (NEB) supplemented with 100 μM sinefungin in a 50 μL reaction at 37° C. for 1 h. After the digestion, the beads were washed twice with 1× B&W buffer (containing 0.01% Tween-20) and three times with 1× B&W buffer. After washings, the beads were dissolved in 30 μL water.

The beads were then used for ligation with 6 μl of Adapter2 (10 μM) using Blunt TA/Ligase Master Mix. The ligation was carried out at 25° C. for 20 min. After the ligation, the beads were washed three times with 1× B&W buffer & subsequently dissolved in 40 μL water.

### Example 29

### Addition of Multiplexing Indexes to Adapter 2 Ligation Products for NGS

The EcoP15I digestion products ligated with Adapter 2 (5 μL beads) were then used for PCR amplification with A1-Rn1-PCR1_i5, A1-Rn1-Fw2_PCR1_i5 and A2-Rn1-Rw1_PCR1_i7 primers for 12 cycles with KAPA HiFi HotStart polymerase. The PCR1 parameters are shown in Table 22.

TABLE 22
PCR1 conditions for addition of sequencing PCR handles to
Adapter 2 ligation products

| Step | | Temperature | Time |
|---|---|---|---|
| Initial Denaturation | | 95° C. | 3 min |
| 12 cycles | Denaturation | 98° C. | 20 s |
| | Annealing | 58° C. | 15 s |
| | Extension | 72° C. | 15 s |

| | | |
|---|---|---|
| Final Extension | 72° C. | 1 min |

The PCR1 products were then purified using ChargeSwitch PCR clean-up beads. These purified PCR1 products (50-100 ng) were used for the addition of multiplexing indexes for 5 PCR cycles with KAPA HiFi HotStart polymerase. For gDNA, defined template 1, defined template 2 and ATAC, different combinations of NEBNext i501-i506 primer and NEBNext i701-i708 primers were used. The PCR2 parameters are mentioned in Table 23. ChargeSwitch PCR clean-up beads were used for PCR2 purification.

TABLE 23
PCR2 conditions for addition of multiplexing indexes to
Adapter 2 ligation products

| Step | | Temperature | Time |
|---|---|---|---|
| Initial Denaturation | | 95° C. | 3 min |
| 5 cycles | Denaturation | 98° C. | 20 s |
| | Annealing | 65° C. | 15 s |
| | Extension | 72° C. | 45 s |

| | | |
|---|---|---|
| Final Extension | 72° C. | 3 min |

The PCR2 products were run on 2% agarose gel. The expected band of 204 bp was observed for Adapter 2 ligated products for gDNA, defined templates (1 and 2) and ATAC (FIG. 8B).

### Example 30

### Ecil Restriction Digestion & Ligation of Adapter 3

The magnetic beads with adapter 2 ligated products from Example 28 (gDNA, ATAC and defined templates 1 and 2) were then digested with Ecil for 1 h at 37° C. in Cutsmart buffer (NEB). After Ecil digestion, the beads were washed thrice with 1× B&W buffer and dissolved in 15 μL water. Ligation of 2-6 μL of Adapter 3 (10 μM) was carried out with Blunt TA/Ligase Master Mix (NEB) for 20 min at 25° C. After the ligation, the beads were washed thrice with 1× B&W buffer and then dissolved in 40 μL water.

### Example 31

### Addition of Multiplexing Indexes to Adapter 3 Ligation Products for NGS

Adapter 3 ligated beads (5 μL) from Example 30 were used for PCR amplification with A1-Rn1-PCR1_15, A1-Rn1-Fw2_PCR1_i5 and Amplify_A3-Rn1-i7 primers for addition of TruSeqA/B PCR handles.

KAPA HiFi Taq polymerase was used for PCR1 with the conditions in Table 24.

TABLE 24
PCR1 conditions for addition of sequencing PCR handles to
Adapter 3 ligation products

| Step | | Temperature | Time |
|---|---|---|---|
| Initial Denaturation | | 95° C. | 3 min |
| 13 cycles | Denaturation | 98° C. | 20 s |
| | Annealing | 57° C. | 15 s |
| | Extension | 72° C. | 15 s |

| | | |
|---|---|---|
| Final Extension | 72° C. | 1 min |

The PCR1 products were purified with ChargeSwitch PCR clean-up beads. These PCR1 products (50-100 ng) were used for addition of multiplexing indexes and amplified for 5 PCR cycles. Combinations of NEBNext i501-1506 primers and NEBNext i701-i708 primers were used. The PCR2 conditions are mentioned in Table 25. PCR2 purification was carried out using ChargeSwitch PCR clean-up beads.

TABLE 25
PCR2 conditions for addition of multiplexing indexes to
Adapter 3 ligation products

| Step | | Temperature | Time |
|---|---|---|---|
| Initial Denaturation | | 95° C. | 3 min |
| 5 cycles | Denaturation | 98° C. | 20 s |
| | Annealing | 65° C. | 15 s |
| | Extension | 72° C. | 45 s |

| | | |
|---|---|---|
| Final Extension | 72° C. | 3 min |

The PCR2 products were run 2% agarose gel and the expected band of 240 bp could be observed (FIG. 8C).

### Example 32

### Acul Restriction Digestion & Ligation of Adapter 4

The beads containing Adapter 3 ligated products (from Example 30) were digested with Acul (NEB) in Cutsmart buffer (NEB) for 1 h at 37° C. in a 30 μL reaction. After the digestion, the enzymatic reaction containing the beads was placed on DynaMag-PCR magnet and the supernatant was transferred to a new tube. The supernatant was then supplemented with 2× Blunt TA/Ligase Master Mix and 1-6 μL of Adapter 4 (10 μM). The ligation was carried out at 25° C. for 20 min. ZYMO DNA Clean & Concentrator kit was used for purification of the ligation reaction.

### Example 33

### Addition of Multiplexing Indexes to Adapter 4 Ligation Products for NGS

The purified ligation products (from Example 32) were then PCR amplified using Amplify_A4-Rn1-i5 and Amplify_A3-Rn1-i7 primers using the PCR1 conditions in Table 26.

TABLE 26
PCR1 conditions for amplification of Adapter 4 ligation products

| Step | | Temperature | Time |
|---|---|---|---|
| Initial Denaturation | | 95° C. | 3 min |
| 14 cycles | Denaturation | 98° C. | 20 s |
| | Annealing | 57° C. | 15 s |
| | Extension | 72° C. | 15 s |

| | | |
|---|---|---|
| Final Extension | 72° C. | 1 min |

PCR1 products were purified with ChargeSwitch PCR clean-up beads and 50-100 ng of them were used for addition of multiplexing indexes using the PCR2 conditions in Table 27. NEBNext i501-i506 primers and NEBNext i701-i708 primers were used for PCR amplification. R2 products were purified with ChargeSwitch PCR clean-up beads.

TABLE 27
PCR2 conditions for addition of multiplexing indexes to
Adapter 4 ligation products

| Step | | Temperature | Time |
|---|---|---|---|
| Initial Denaturation | | 95° C. | 3 min |
| 5 cycles | Denaturation | 98° C. | 20 s |
| | Annealing | 65° C. | 15 s |
| | Extension | 72° C. | 45 s |

| | | |
|---|---|---|
| Final Extension | 72° C. | 3 min |

These PCR2 products were then run on 2% agarose gel and expected band of 270 bp was observed (FIG. 8D).

### Example 34

### Sequencing on Miseq

The sequencing libraries generated from Example 26, Example 29, Example 31 and Example 33 were pooled and sent for sequencing using Miseq v2 300 bp paired kit.

### Example 35

### Data Analysis of NGS

The sequencing reads for adapter 4 ligated products (Example 33) were trimmed for the adapter sequences using cutadapt 4.1 and the reads shorter than 17 bp and longer than 25 bp were discarded. The filtered reads between 17-25 bp were aligned to the human genome hg38 using Bowtie2. Samtools was used to convert SAM to BAM files. Bedtools was used for intersecting DNAse hypersensitivity regions with ATAC and gDNA reads. UCSC Genome browser was used to visualize alignment of reads to the genome.

### Results

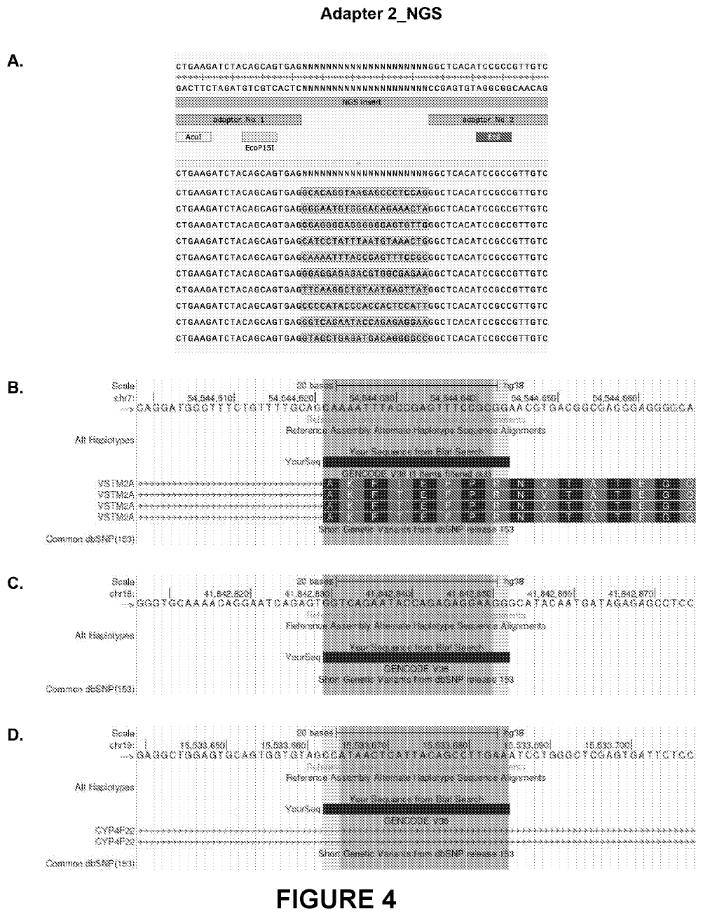

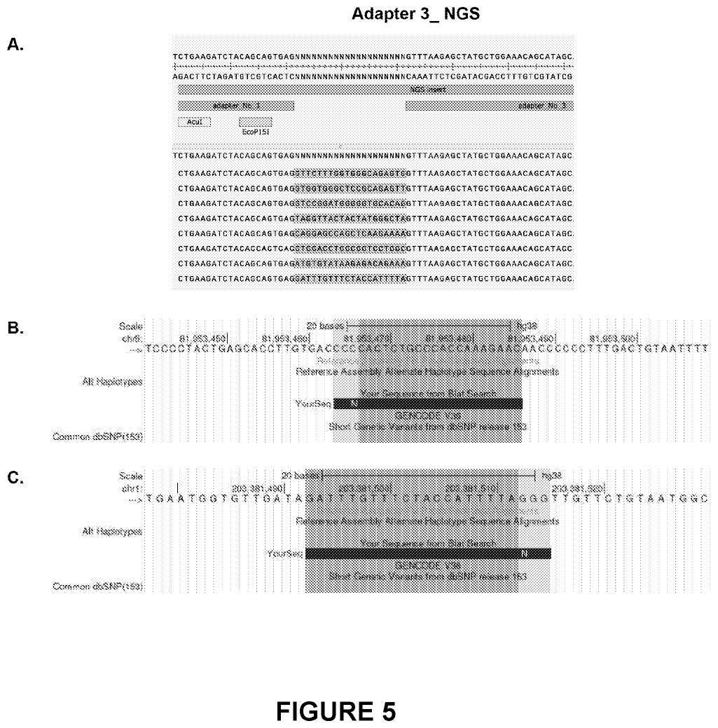

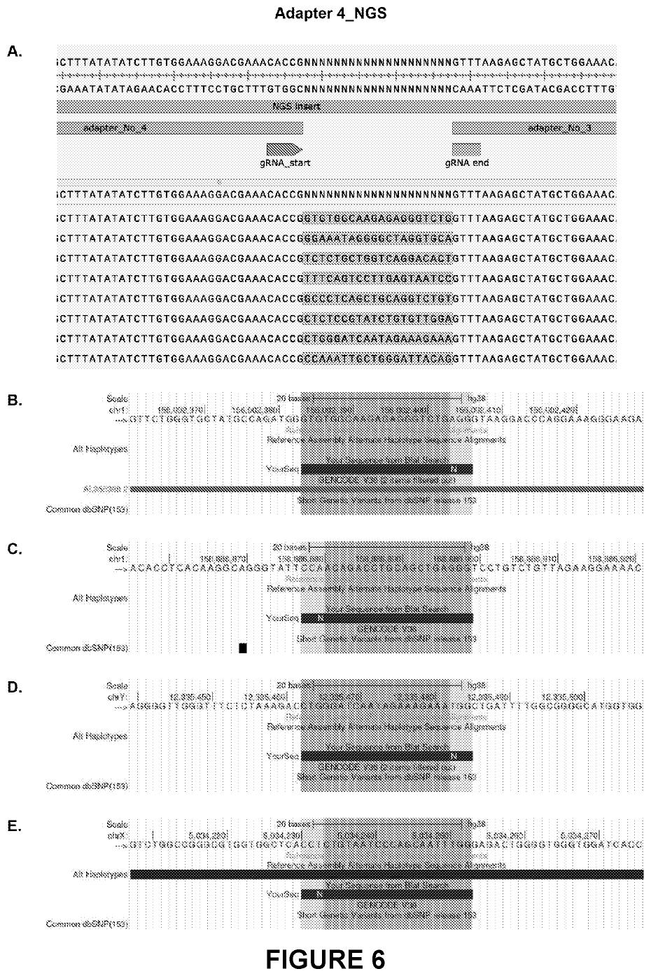

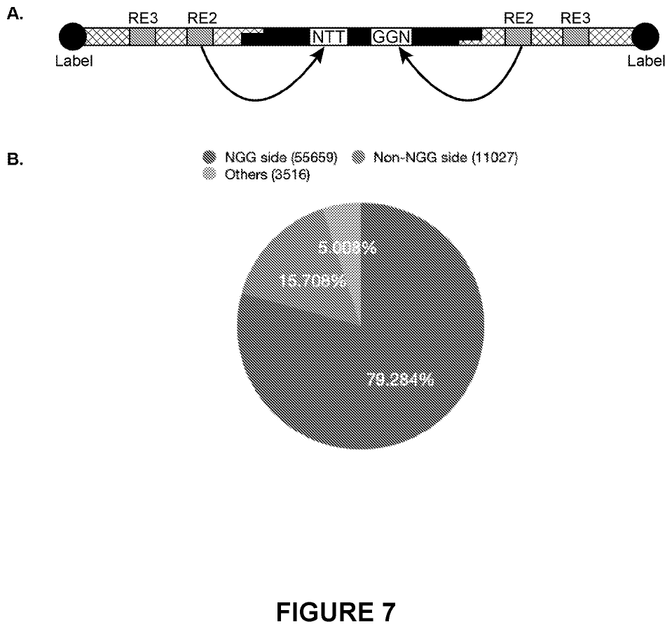

The sequencing of ligation products of Adapter 2 (FIG. 4), Adapter 3 (FIG. 5) and Adapter 4 (FIG. 6) for gDNA sample showed the correct orientation of inserts and these could be aligned back to the genome. One of the defined templates had NGG on one end and NTT on the other end. NGG bias could be clearly observed after sequencing (FIG. 7) demonstrating the specificity of the method.

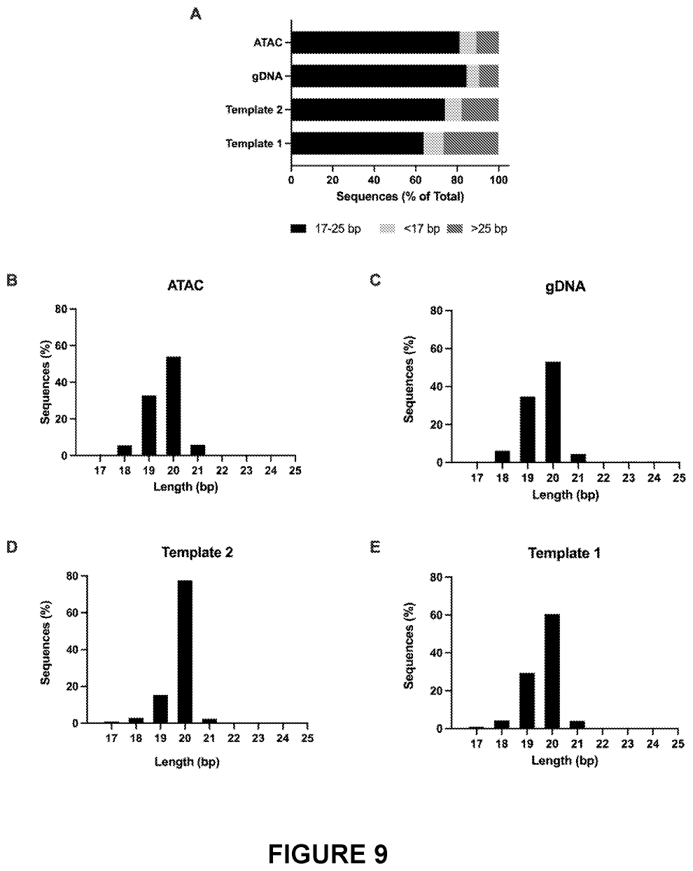

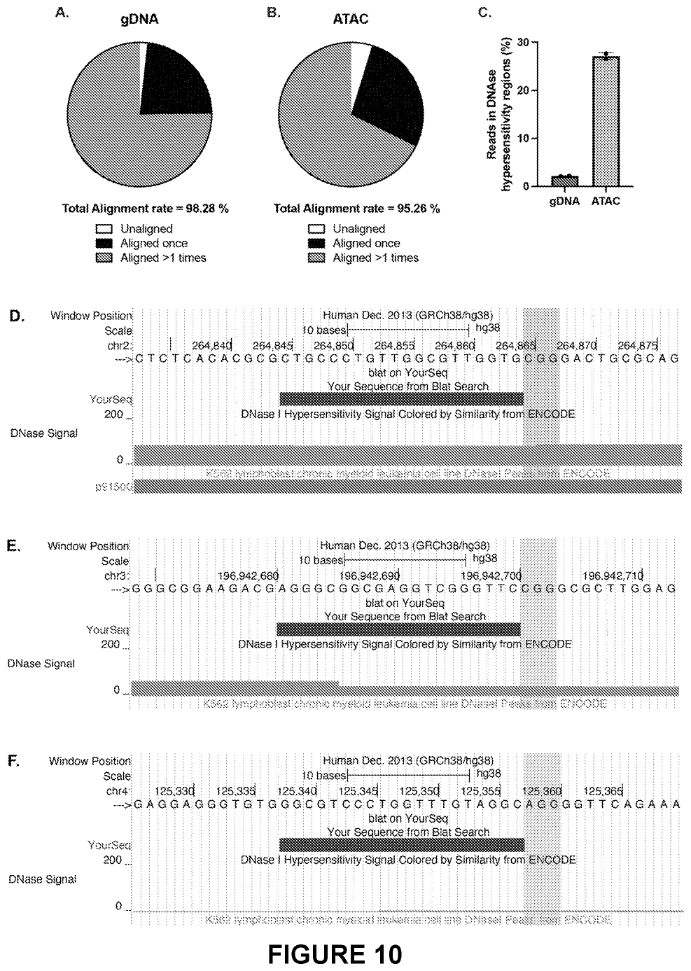

The percentage of total sequencing reads for adapter 4 ligated products (Example 33) after adapter trimming that were of the correct length (17-25 bp) is shown in FIG. 9A, along with the sequences that were discarded from analysis (<17 bp and >25 bp). The length distribution of the filtered reads (17-25 bp) is shown in FIG. 9(B-E) and most of the reads were 20 bp long followed by 19 bp reads. Upon alignment to the genome using Bowtie2, most of the reads were aligned (FIG. 10A and 10B).

The sequencing reads for gDNA and ATAC samples for adapter 4 ligated products (from Example 33) were intersected with DNAse hypersensitivity regions in the genome using bedtools. Sequencing reads from the ATAC sample are present 12-fold higher than gDNA in DNAse hypersensitivity regions (FIG. 10C). Finally, when ATAC reads from Adapter 4 ligated products (Example 33) were aligned to the genome using UCSC genome browser, the presence of NGG PAM can be found (FIG. 10D-10F).

1. A method of generating a library of polynucleotide molecules encoding guide RNAs (gRNAs) from target polynucleotide(s) comprising incubation of the target polynucleotide(s) with insertional enzyme complexes, wherein each of said insertional enzyme complexes comprises (i) an insertional enzyme and (ii) one or more tagmentation adapters to generate a plurality of tagged cleavage fragments.

2. The method of claim 1, which comprises the following steps:
(a) incubation of the target polynucleotide(s) with insertional enzyme complexes comprising (i) an insertional enzyme and (ii) one or more tagmentation adapters which comprise a first restriction site, to generate a plurality of adapter-attached polynucleotide fragments;
(b) amplification of the product of step (a);
(c) restriction digestion of the product of step (b) with a restriction enzyme which recognises the first restriction site and cleaves the adapter-attached polynucleotide fragment at a site downstream so as to remove the tagmentation adapters from the polynucleotide fragment;
(d) ligation of the digested product of step (c) to a plurality of first ligation adapters which comprise a second restriction site, a third restriction site, and a label, to generate a plurality of adapter-attached polynucleotide fragments wherein each polynucleotide fragment is flanked by two first ligation adapters;
(e) restriction digestion of the product of step (d) with a restriction enzyme which recognises the second restriction site and cleaves the adapter-attached polynucleotide fragments within the polynucleotide region to generate a plurality of adapter-attached polynucleotide fragments comprising a first ligation adapter, a crRNA sequence and part or all of a protospacer adjacent motif (PAM) sequence;
(f) ligation of the digested product of step (e) to a plurality of second ligation adapters which comprise a fourth restriction site and either part or all of a PAM sequence to generate a plurality of adapter-attached polynucleotide fragments wherein the polynucleotide fragment is flanked by a first ligation adapter and a second ligation adaptor;
(g) restriction digestion of the product of step (f) with a restriction enzyme which recognises the fourth restriction site and cleaves the adapter-attached polynucleotide fragment at a site downstream so as to remove the second ligation adaptor and the PAM sequence;
(h) restriction digestion of the product of step (g) with a restriction enzyme which recognises the third restriction site and cleaves the adapter-attached polynucleotide fragment at a site downstream so as to remove the first ligation adaptor, generating a plurality of gRNAs; and
(i) ligation of the digested product of step (h) to a plurality of third and fourth ligation adapters which comprise vector cloning sequences, to generate a plurality of adapter-attached gRNAs.

3. The method of claim 2, wherein step (a), (b), (c), (d), (e), (f), (g), (h) and/or step (i) additionally comprise a purification step.

4. The method of claim 3, wherein the purification step is performed by magnetic separation.

5. The method of claim 3, wherein the purification step is performed by absorption onto a solid matrix.

6. The method of claim 2, wherein step (d), step (f) and/or step (i) additionally comprise amplification of the adapter-attached oligonucleotides.

7. The method of claim 2, wherein the first restriction site is an Mmel restriction site.

8. The method of claim 2, wherein the restriction enzyme which recognises the first restriction site is Mmel.

9. The method of claim 2, wherein the second restriction site is an EcoP15I restriction site.

10. The method of claim 2, wherein the restriction enzyme which recognises the second restriction site is EcoP15I.

11. The method of claim 2, wherein the third restriction site is an Acul restriction site.

12. The method of claim 2, wherein the restriction enzyme which recognises the third restriction site is Acul.

13. The method of claim 2, wherein the fourth restriction site is an Ecil restriction site.

14. The method of claim 2, wherein the restriction enzyme which recognises the fourth restriction site is Ecil.

15. The method of claim 2 wherein the PAM sequence is 5′-NGG-3′, where N can be any nucleotide base.

16. The method of claim 2, wherein the insertional enzyme is Tn5 transposase or a variant or fusion Tn5 transposase thereof.

17. A library of polynucleotide molecules encoding guide RNAs (gRNAs) obtainable by the method of claim 2.

18. A guide RNA (gRNA) library generation kit which comprises:
(a) an insertional enzyme;
(b) a plurality of tagmentation adapters which comprise a first restriction site;
(c) a restriction enzyme which recognises the first restriction site;
(d) a plurality of first ligation adapters which comprise a second restriction site, a third restriction site, and a label;
(e) a restriction enzyme which recognises the second restriction site;
(f) a plurality of second ligation adapters which comprise a fourth restriction site and part or all of a protospacer adjacent motif (PAM) sequence;
(g) a restriction enzyme which recognises the fourth restriction site;
(h) a restriction enzyme which recognises the third restriction site; and
(i) a plurality of third and fourth ligation adapters which comprise vector cloning sequences.

19. The kit of claim 18, wherein:
(a) the first restriction site is an Mmel restriction site; and/or
(b) the restriction enzyme which recognises the first restriction site is Mmel; and/or
(c) the second restriction site is an EcoP15I restriction site; and/or
(d) the restriction enzyme which recognises the second restriction site is EcoP15I; and/or
(e) the third restriction site is an Acul restriction site; and/or
(f) the restriction enzyme which recognises the third restriction site is Acul; and/or
(g) the fourth restriction site is an Ecil restriction site; and/or
(h) the restriction enzyme which recognises the fourth restriction site is Ecil; and/or
(i) the PAM sequence is 5′-NGG-3′, where N can be any nucleotide base; and/or
(j) the transposase is Tn5 transposase.
